(19) United States
(12) Patent Application Publication
(10) Pub. No.: US 2009/0136466 A1
(43) Pub. Date: May 28, 2009

(54) METHODS FOR MAKING SKIN CELL DERIVED STEM CELLS

(75) Inventors: Bernd Fritzsch, Omaha, NE (US); Kirk Beisel, Omaha, NE (US)

Correspondence Address: POLSINELLI SHUGHART PC, 700 W. 47TH STREET, SUITE 1000, KANSAS CITY, MO, 64112-1802, US

(73) Assignee: Creighton University, Omaha, NE (US)

(21) Appl. No.: 12/275,860

(22) Filed: Nov. 21, 2008

Related U.S. Application Data
(60) Provisional application No. 60/989,727, filed on Nov. 21, 2007.

(51) Int. Cl. A61K 35/12; C12N 5/00; A61P 27/16; C12N 15/09

(52) U.S. Cl. 424/93.21; 435/325; 435/455

(57) ABSTRACT

The present invention relates to methods of deriving a stem cell from a skin cell.

### CROSS REFERENCE TO RELATED APPLICATIONS

This application claims the priority of U.S. provisional application No. 60/989,727, filed Nov. 21, 2007, which is hereby incorporated by reference in its entirety.

### FIELD OF THE INVENTION

The invention encompasses methods of making stem cells derived from skin cells.

### BACKGROUND OF THE INVENTION

Recent scientific developments have shown the potential usefulness of human stem cells. Sources of human stem cells may be controversial, however. Typically, human stem cells are derived from embryonic stem cells, and this raises potential ethical issues. Consequently, other sources of stem cells would provide a valuable addition to the current stem cell resources.

### SUMMARY OF THE INVENTION

One aspect of the present invention encompasses a vector that comprises at least one stem cell specific gene operably linked to a promoter. The promoter's activity is sensitive to the concentration of an activator.

Another aspect of the invention encompasses a composition comprising at least one first vector. The first vector comprises at least one stem cell specific gene operably linked to a promoter, wherein the promoter's activity is sensitive to the concentration of an activator. The composition also includes at least one second vector that comprises a recombinase.

Yet another aspect of the invention encompasses a skin cell that comprises at least one chromosomally integrated stem cell specific gene operably linked to a promoter. The promoter's activity is sensitive to the concentration of an activator.

Still another aspect encompasses an ear cell generated from a skin cell derived stem cell. The ear cell comprises at least one chromosomally integrated stem cell specific gene operably linked to a promoter. The promoter's activity is sensitive to the concentration of an activator.

Yet still another aspect of the invention encompasses a method for generating a skin cell derived stem cell. The method comprises integrating at least one stem cell specific gene into the chromosomal DNA of a skin cell. The stem cell specific gene is operably linked to a promoter, wherein the promoter's activity is sensitive to the concentration of an activator. The stem cell specific gene is expressed to generate a skin cell derived stem cell.

An additional aspect of the invention encompasses a method for generating an ear cell from a skin cell derived stem cell. The method comprises inducing the skin cell derived stem cell to differentiate into an ear cell.

Another additional aspect of the invention encompasses a method for generating an ear cell from a skin cell. The method comprises integrating at least one stem cell specific gene into the chromosomal DNA of the skin cell. The stem cell specific gene is operably linked to a promoter, wherein the promoter's activity is sensitive to the concentration of an activator. The stem cell specific gene is expressed to generate a skin cell derived stem cell, and induced to differentiate into an ear cell.

Yet another additional aspect of the invention encompasses a method for generating an ear cell from a skin cell. The method comprises integrating at least one stem cell specific gene into the chromosomal DNA of a skin cell. The stem cell specific gene is operably linked to a promoter, wherein the promoter's activity is sensitive to the concentration of an activator. An activator is administered to induce expression of the stem cell specific gene thereby generating a skin cell derived stem cell. The administration of the activator is stopped and the skin cell derived stem cell is induced to differentiate into an ear cell.

An alternative aspect of the invention encompasses a method for decreasing hearing loss in a subject. The method comprises administering a skin cell derived stem cell to the subject.

An additional alternative aspect of the invention encompasses a method for decreasing hearing loss in a subject. The method comprises administering an ear cell generated from a skin cell derived stem cell to the subject.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

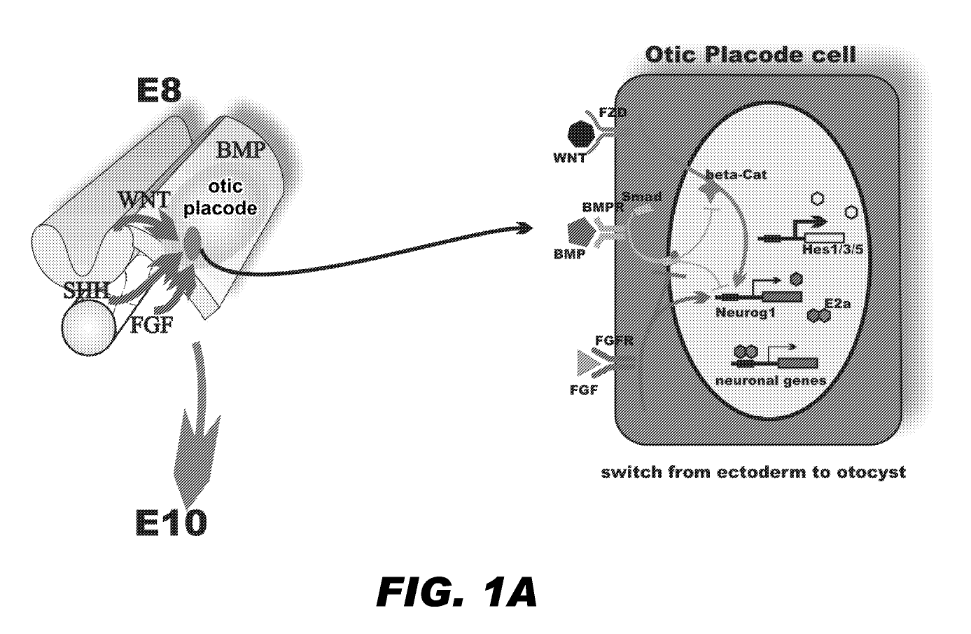

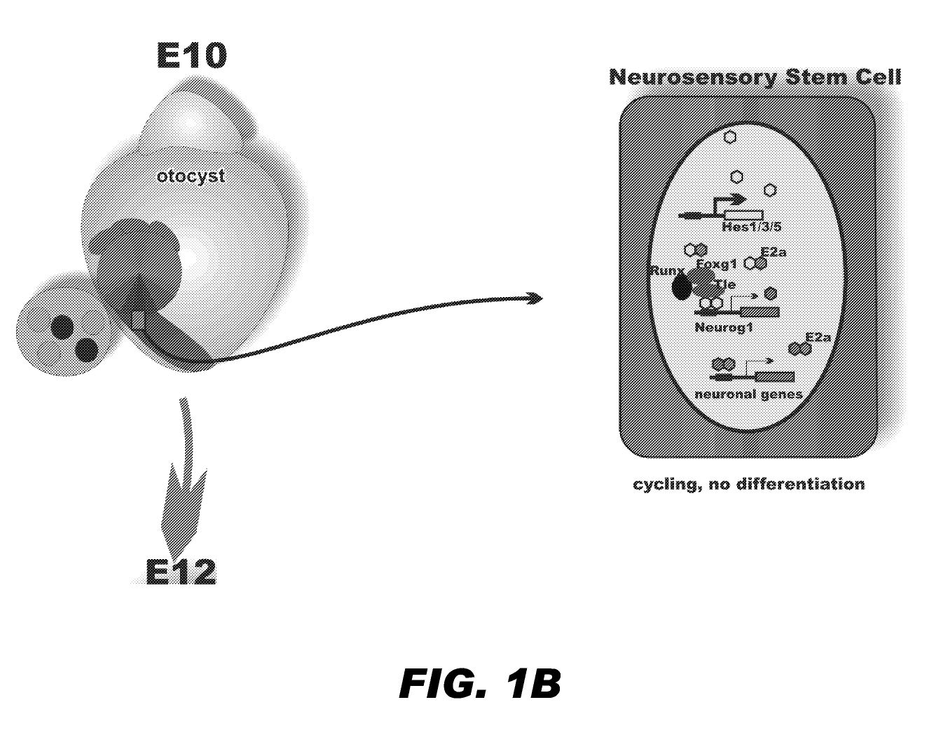

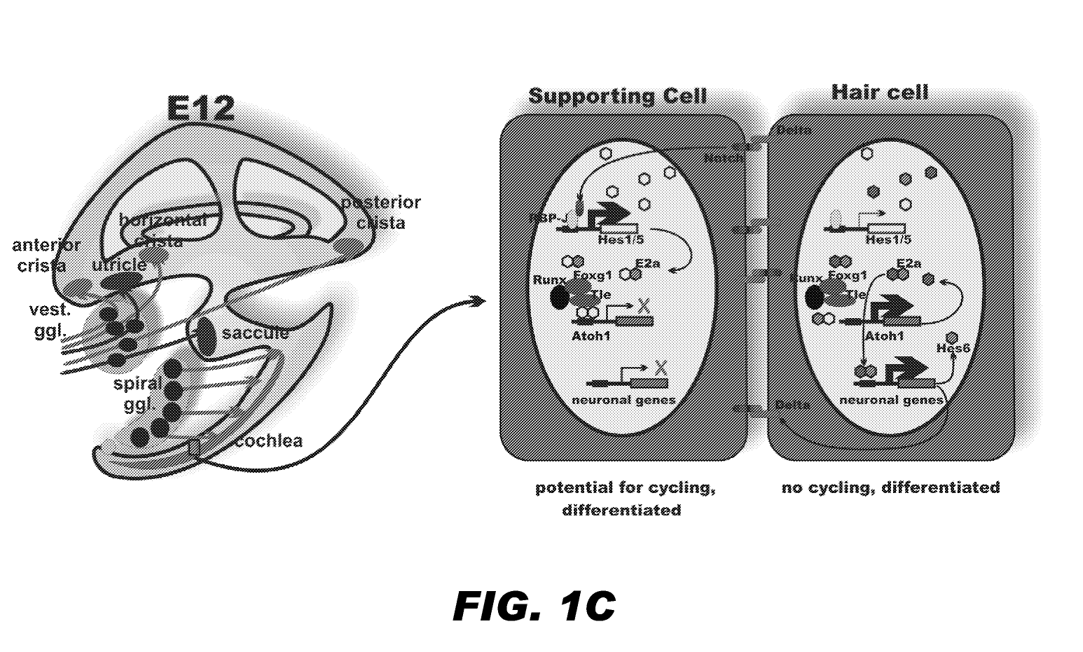

FIG. 1 illustrates organ, cell, and molecular interactions in ear development. The morphogenesis (left) and some molecular interactions underlying proliferation and cell fate decision (right) are depicted in this scheme. Morphogenesis transforms a small patch of ectoderm between embryonic days 8 and 12 into a complex labyrinth of ducts and recesses that harbors the six sensory epithelia of the mammalian ear in strategic positions for extraction of epithelia-specific energy. Delamination of sensory neurons generates the vestibular and cochlear sensory neurons that connect specific sensory epithelia of the ear to specific targets in the hindbrain. One of the earliest steps in this process is the selection of otic placode cells through the interaction of several diffusible factors; in particular, FGF and WNT signaling upregulates both inhibitory and activating bHLH genes to switch the cell fate through down regulation of BMP signaling, specifying the position and size of the otic placode (top right). These stem cells will, through the interaction of activator- and inhibitor-type bHLH genes remain in cycling phase without differentiation resulting in clonal expansion. As cells progress through the cycles, they will change their fate determination, giving rise to neurosensory stem cells (middle right) that form by asymmetric divisions all sensory neurons of the ear. Some neurosensory stem cells as well as independently arising cells of the otic placode turn into sensory epithelia precursor cells (SNP). These cells will give rise by asymmetric divisions to hair cells and supporting cells (bottom right). Exit from the cell cycle, combined with proper cell fate specification to, e.g., hair cell and supporting cell, will be mediated in part by the NOTCH-reinforced switch to either explosive upregulation of proneuronal bHLH genes (Atoh1 in the case of hair cells) or of inhibitory bHLH genes (such as Hes1 or Hes5) by the γ-secretase-cleaved Notch fragment that binds to RBPSUH (formerly Rbp-J). The action of HES homodimers on N-boxes to turn on proneuronal genes is enhanced through interaction with the TLE, RUNX, FOXG and genes. Consequently, eliminating for example Foxg1 results in diminished efficacy of HES signaling resulting in premature cell cycle exit and differentiation. Shortly after E14, all proliferative activity in the PNP progenitors stops and no new sensory neurons or hair cells will form. Modified after Refs 37,38.

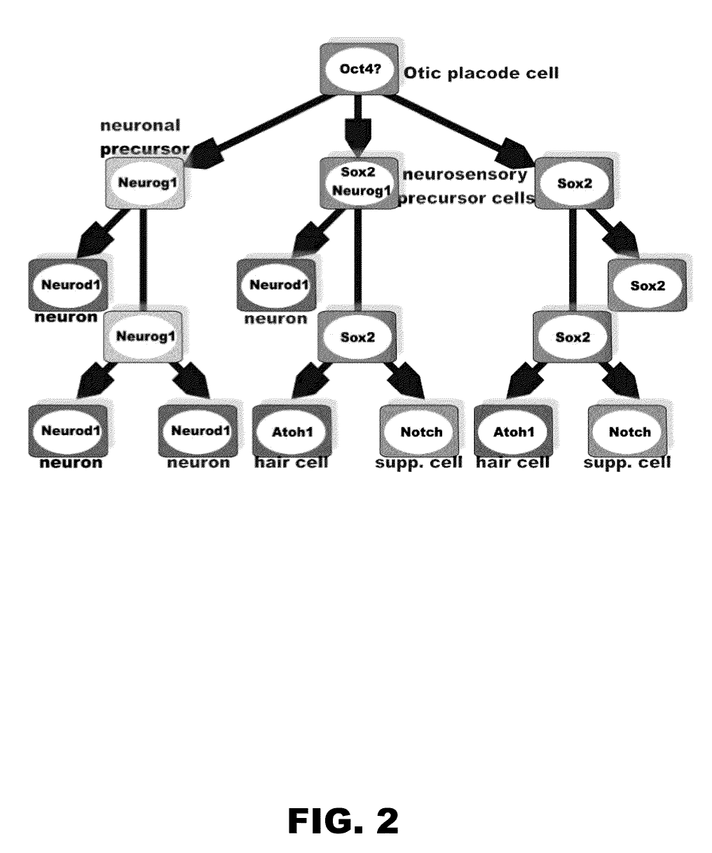

FIG. 2 presents an overview of cell-type-specific and overlapping precursors. Analysis of several null mutations suggest that there is an initial formation of two, partially overlapping, precursor populations, a neuronal precursor characterized by Neurog1 expression and a neurosensory precursor, characterized by Sox2 expression. The 40-80% reduction of hair cell and supporting cell formation in Neurog1 null mice suggests that the size of the common neuronal/neurosensory precursor population varies in different sensory epithelia. The later-expressed bHLH gene Neurod1 does not show this massive effect on hair cells and appears to be exclusively expressed in differentiating neurons. Absence of hair cell differentiation in Sox2 and Atoh1 null mice suggests that these genes are essential for hair cell formation, no matter what origin. Supporting cells depend on the hair-cell-mediated upregulation of Notch (and Hes) for their differentiation and will turn into hair cells in the absence of proper Notch/Hes signaling. Modified after Refs 39,49,61,64,120.

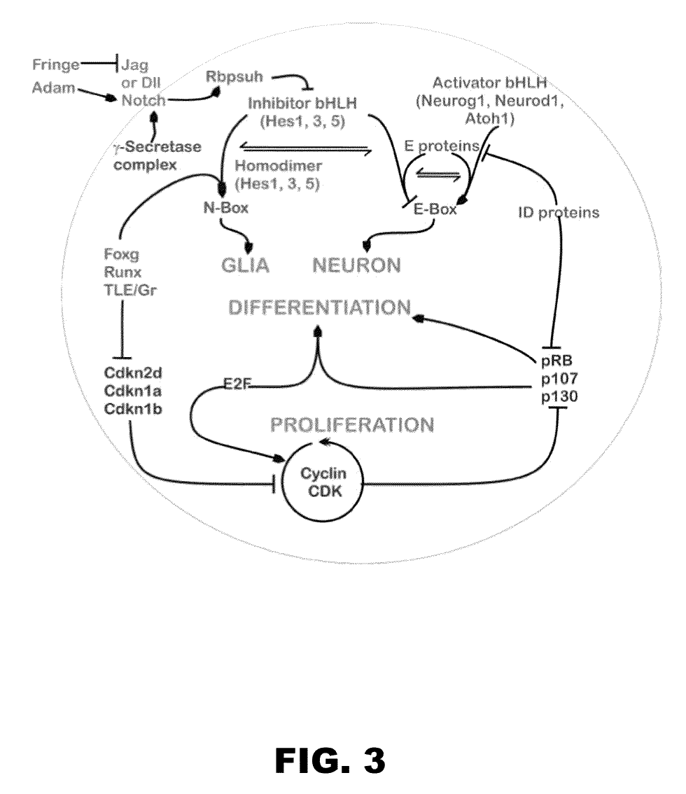

FIG. 3 depicts key signaling pathways for inner ear proliferation and differentiation. This schematic diagram represents an overview of the known and presumed interactive pathways for proliferation and differentiation of the neurosensory cells in the inner ear. Signaling of the membrane bound (brown) Notch receptors by binding to their ligands, Delta and Jagged, can be influenced by the extracellular (purple) Fringe and ADAM enzymes. Fringe inhibits (blocked line) the Notch binding of Jagged, while Adam cleaves the Notch receptor to potentate its activation (lined arrow). The cleavage of intercellular domain fragment of Notch is done by the cytoplasmic (dark blue) γ-secretase complex which then activates the nuclear protein (red) RBPSUH. Inactivated RBPSUH blocks transcription of the Hes genes whereas activation enhances transcription. Homodimers of HES proteins can bind to N-boxes to initiate differentiation (green) of glial precursors. N-box binding of HES homodimers is regulated further by a FOXG, RUNX and TLE promoter complex. Heterodimers between HES and E proteins bind and competitively block usage of E-box-binding sites. Activation of E-box promoter sequences is through the combined E-protein and the activator bHLH heterodimers and this permits neuronal differentiation. To do this, the activator bHLH proteins compete with HES proteins for the E-protein-binding partners. E proteins can also be inactivated from DNA binding through interaction with the inhibitor of DNA-binding (ID) proteins, which also suppress the cell cycle (blue) retinoblastoma isoforms. The pRB isoforms alone or in combination E2F proteins cause cell differentiation. Cell proliferation (green) is mediated through the proteins of cyclin CDK pathway that phosphorylate Rb to allow E2F proteins to initiate the S-phase entry. The cyclin CDK proteins can also inhibit differentiation via pRB phosphorylation, whereas cyclin dependent kinase inhibitors (Cdkn) prevent proliferation. Expression of the CDKNs is blocked by the FOXG, RUNX and TLE complex, allowing differentiation of glia cells through enhanced action of HES homodimers on the N-Box. Modified after Refs 11,46,78,85.

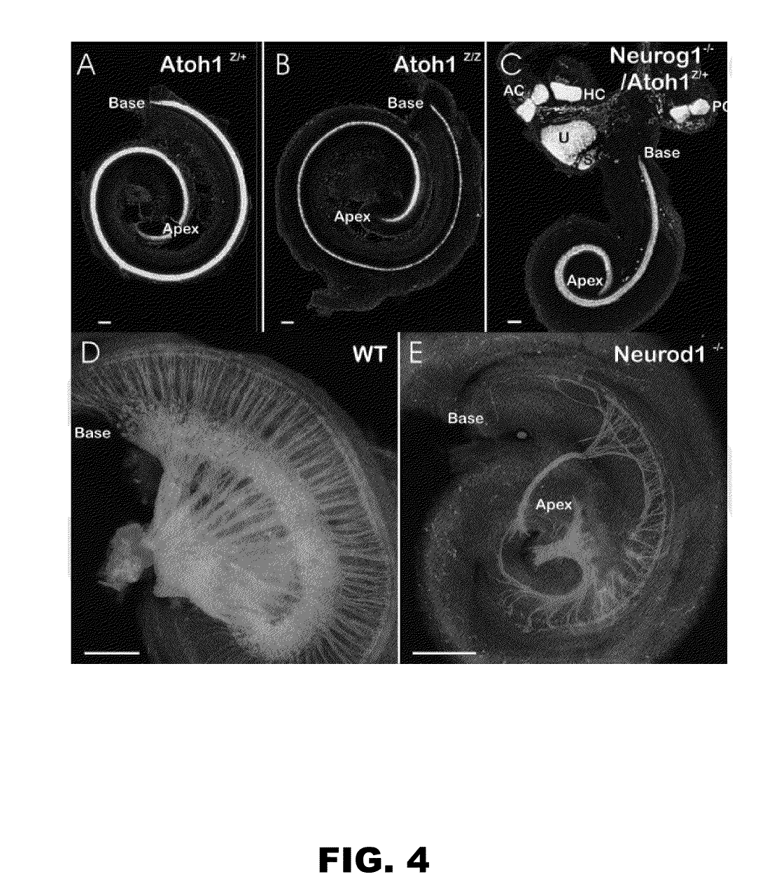

FIG. 4 depicts examples of gene effects on histogenesis and morphogenesis. A,B,D,E: Flat-mounted cochlea or C: entire ears show the effects of targeted deletion of an activator-type bHLH gene (Atoh1, B; Neurog1,C; Neurod1,E) on the presence of hair cells (revealed by Atoh1-lac Z expression in A-C) or innervation (revealed by lipophilic dye tracing in D,E). Note that both the distribution of Atoh1-lac Z positive cells as well as the overall length of the cochlea (base and apex are indicated) show little difference in Atoh1-lac Z heterozygote and null mutants, despite the fact that no hair cells differentiate in Atoh1 null mice. This suggests that the late upregulation of a bHLH gene in cells destined to exit the cell cycle is of little consequence for morphogenesis and cellular patterning in the ear. In contrast, earlier upregulated bHLH genes such as Neurog1 (C) or Neurod1 (E) have a more profound morphogenetic effect such as shortening of the cochlea (C,E) or almost complete loss of sensory epithelia (saccule in E). Additional effects are displaced development of some hair cells outside the typical sensory epithelia (C) or loss of a large fraction of sensory neurons combined with an alteration in the pattern of innervation. Modified after Refs 39,64,68. AC, anterior crista; HC, horizontal crista; PC, posterior crista, S, saccule; U, utricle. Bar indicates 100 μm.

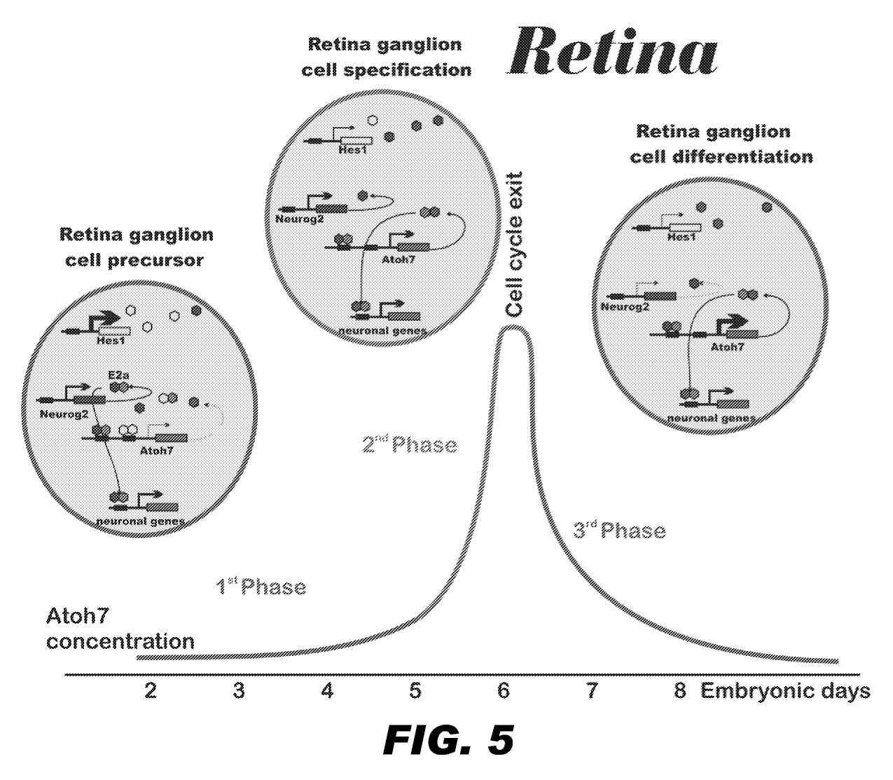

FIG. 5 depicts bHLH gene interactions in retinal ganglion cell specification. The most-detailed single-cell quantitative PCR analysis shows that relative concentrations of bHLH transcripts vary systematically during chicken retina ganglion cell formation. In the first phase (red line), Hes1 transcript exceeds that of Neurog2 and very much that of Atoh7. This dominance of inhibitory bHLH gene expression will result in homodimers on N-boxes (yellow hexagons) as well as few heterodimers of Neurog2 with E2a on E-boxes (lilac/blue hexagons). In phase 2 (blue lines) Hes1 is down regulated allowing Atoh7 transcript to become as prominent as Neurog2 and to form heterodimers with E2a proteins to bind to specific E-boxes (red and blue hexagons). In the third phase (green line) Atoh7 is further upregulated to drive ganglion cell differentiation as well as preventing the developing neuron from reentering the cell cycle. Modified after Ref 109.

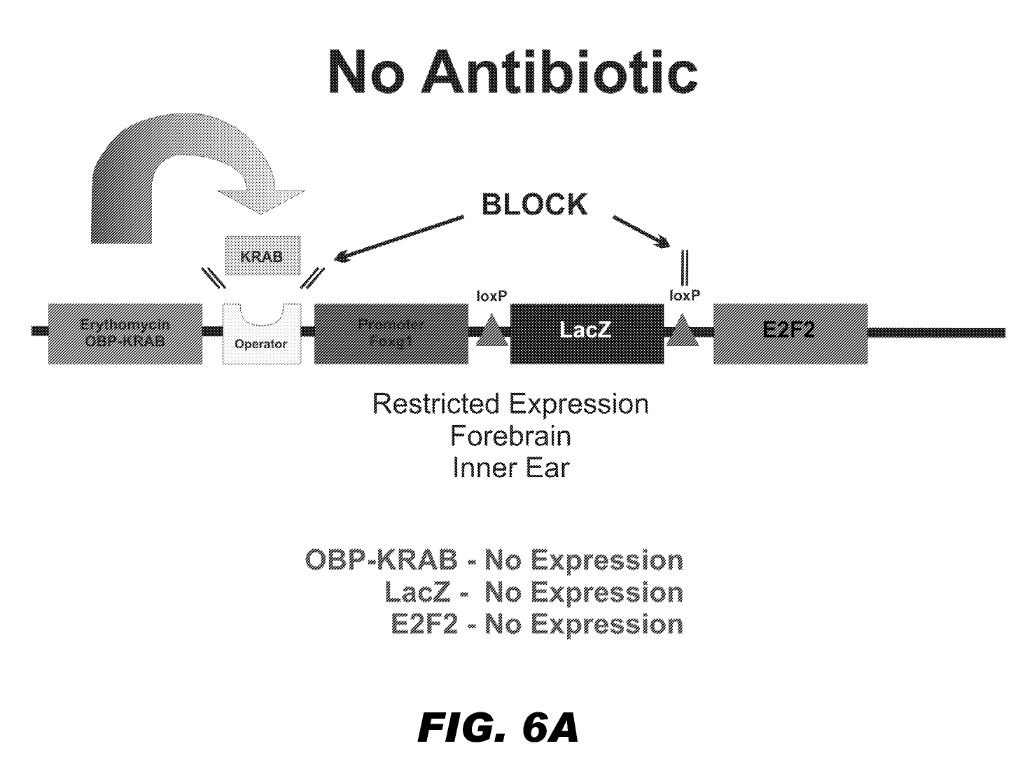

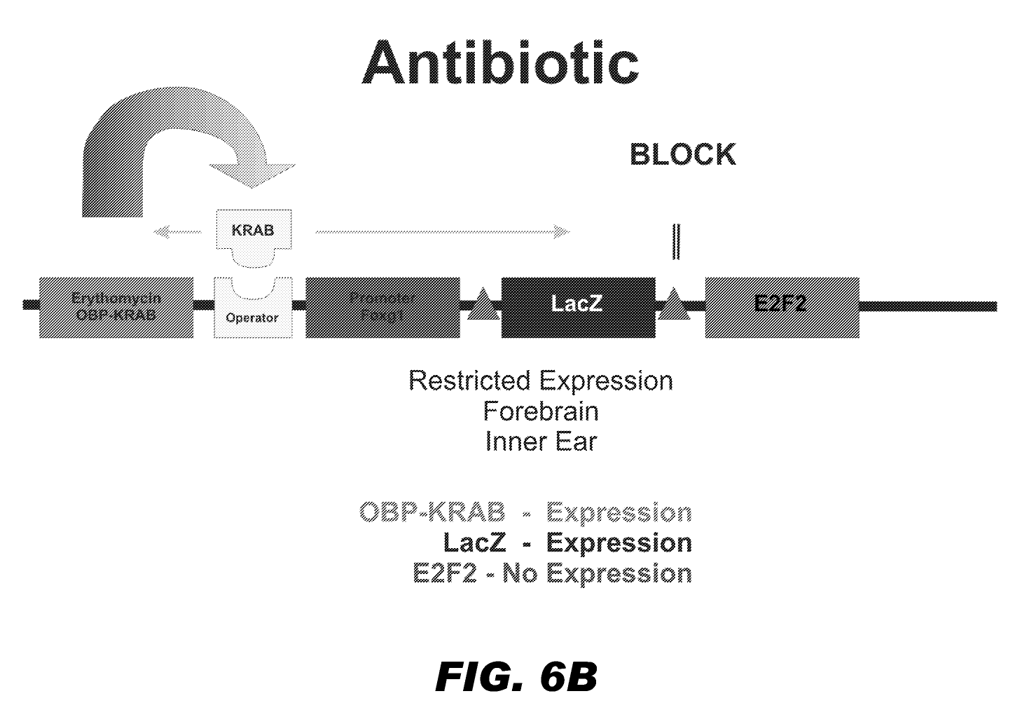

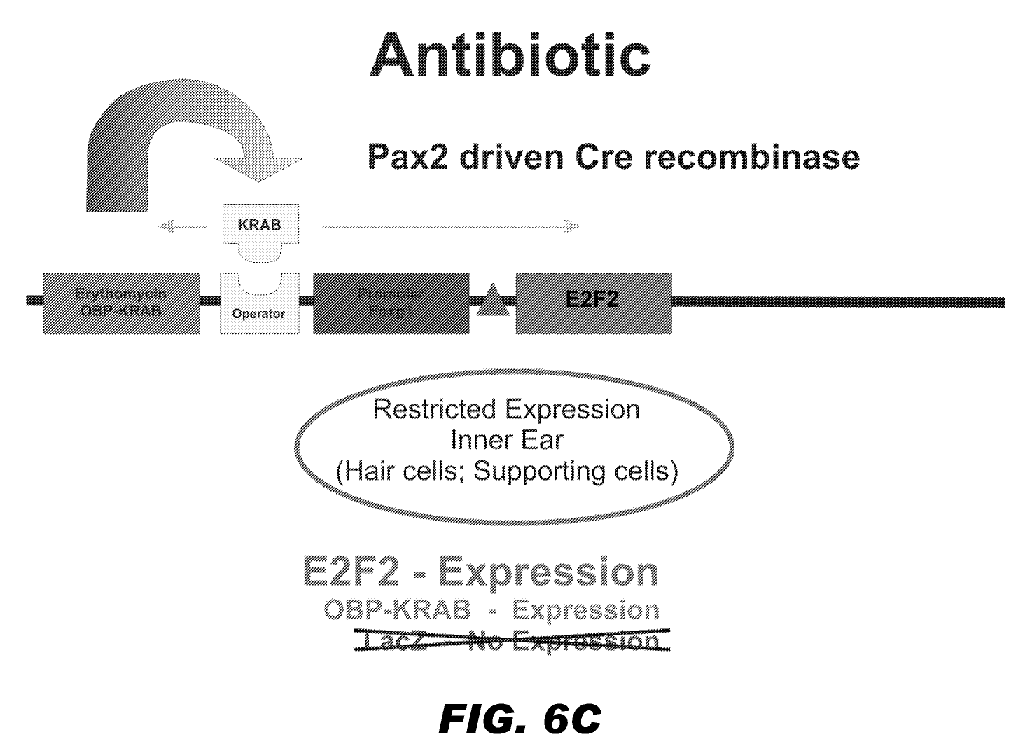

FIG. 6 depicts an illustration of vector comprising a promoter wherein the promoter's activity is sensitive to the concentration of an activator.

### DETAILED DESCRIPTION OF THE INVENTION

The present invention encompasses methods for generating skin derived stem cells. As used herein, the term “skin derived stem cell” refers to a stem cell that is generated from a skin cell. As used herein, stem cell refers to a cell that has the ability to self-replicate, thereby producing more stem cells, as well as the ability to produce progeny cells that differentiate into other types of cells. Stem cells may be pluripotent, i.e., can develop into most cell types, multipotent, i.e., can develop into several cell types, or unipotent, i.e., can develop into one cell type. In other words, a skin cell derived stem cell is a de-differentiated skin cell. A skin derived stem cell may be pluripotent, mutipotent, or unipotent.

In certain embodiments, the invention provides a vector composition comprising a first and second vector. Generally speaking, the vector system comprises at least one stem cell specific gene, that when expressed in a skin cell, aids in de-differentiating the skin cell to a stem cell, which can then be directed to differentiate into a different cell type.

### I. Vector Comprising at Least One Stem Cell Specific Gene

One aspect of the present invention is a vector that comprises at least one stem cell specific gene. The stem cell specific gene is operably linked to a promoter. Generally speaking, the promoter's activity is sensitive to the concentration of an activator.

### (a) Stem Cell Specific Gene

As used herein, the term “stem cell specific gene” refers to a nucleic acid sequence that encodes a gene product found in stem cells. Non-limiting examples of stem cell specific genes may include Oct4 (also known as Pou5f1), Nanog, Sox2, GATA3, Neurog1, KLF4, c-MYC, and LIN28. A vector of the invention may comprise at least one stem cell specific gene, at least two stem cell specific genes, at least three stem cell specific genes, at least four stem cell specific genes, or at least five stem cell specific genes.

The stem cell specific genes may be operably linked to the promoter individually or in tandem. Tandem, as used herein, refers to more than one stem cell specific gene operably linked to a single promoter.

The methods and techniques for preparing vectors are well known in the art. For instance, see Molecular Cloning: A Laboratory Manual, 3rd edition, David W. Russell and Joe Sambrook (2001), Cold Spring Harbor Press and the examples.

### (b) Promoter

Generally speaking, the nucleic acid sequence of the stem cell specific gene is operably linked to a promoter. The term operably linked, as used herein, may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term promoter, as used herein, may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents (i.e. an inducible promoter). Non-limiting representative examples of promoters may include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter. Additionally, the promoter may be a CMV immediate early promoter/enhancer (PCMV) or the CMV enhancer/chicken β-actin promoter (pCAG).

Generally speaking, the promoter should be selected based on the strength of the promoter, the temporal control of the promoter, and the spatial control of the promoter. In some embodiments, the promoter is organ specific. In other embodiments, the promoter is tissue specific. In some embodiments, the promoter is cell specific. For instance, the promoter may be specific for supporting cells in the inner ear. Non-limiting examples of a supporting cell specific promoter are the PLP promoter and the Gfap promoter.

In some embodiments, the promoter is sensitive to the concentration of an activator. For instance, the promoter may be inactive in the absence of the activator. Similarly, the activity of the promoter may increase with an increasing concentration of the activator. Suitable activators may include antibiotics. For instance, an activator may be tetracycline, a streptogramin (for instance, erythromin), or a macrolide (for instance, pristinamycin).

In some embodiments, the promoter is a promoter described in Biotechnology and Bioengineering (2003) 83(7):810-20, hereby incorporated by reference in its entirety.

### (c) Recombinase Site

The vector may further comprise a recombinase site. Generally speaking, a recombinase site is nucleic acid sequence recognized by a recombinase, such as an integrase. One skilled in the art is aware that the selection of a recombinase site depends on the recombinase being used. Typically, a recombinase site may be recognized by a tyrosine recombinase (i.e. a recombinase that uses a tyrosine-mediated mechanism of recombination) or a serine recombinase (i.e. a recombinase that uses a serine-mediated mechanism). Tyrosine recombinases are well known in the art, and include Cre and FLP. Serine recombinases are also well known in the art, and include phage integrases, such as the φC31 integrase. In one embodiment, the serine recombinase site is a site recognized by a φC31 integrase. For instance, the site may be an attB, attP, attL, or attR site. The nucleic acid sequence of an attB, attP, attL or attR site is known in the art.

### II. Composition Comprising at Least One First Vector and at Least One Second Vector

The present invention provides, in part, a composition comprising at least one first vector and at least one second vector. The first vector comprises, in part, the nucleic acid sequence of a stem cell specific gene. The second vector comprises, in part, the nucleic acid sequence of a recombinase. The vectors are designed so that, generally speaking, when a cell is contacted with both vectors, the nucleic acid sequence of the stem cell specific gene will be integrated into the chromosomal DNA of the cell, and the cell will subsequently express the stem cell specific gene. Advantageously, the cell may indefinitely (as opposed to transiently) express the stem cell specific gene.

The methods and techniques for preparing vectors are well known in the art. For instance, see Molecular Cloning: A Laboratory Manual, 3^{rd} edition, David W. Russell and Joe Sambrook (2001), Cold Spring Harbor Press. Each vector is described in more detail herein.

### (a) First Vector

The first vector, as detailed in Section I above, comprises in part the nucleic acid sequence of a stem cell specific gene. Generally speaking, the nucleic acid sequence of the stem cell specific gene is operably linked to a promoter. Additionally, the first vector may comprise a recombinase site. Typically, but not necessarily, the first vector may also comprise a polyadenylation signal. Suitable polyadenylation signals may include the SV40 polyadenylation sequence. Usually, the polyadenylation signal is located 3′ to the stem cell specific gene.

### (b) Second Vector

The second vector comprises in part a nucleic acid sequence of a recombinase. The recombinase may be operably linked to a promoter. Typically, but not necessarily, the second vector may also comprise a polyadenylation signal. Suitable polyadenylation signals may include the SV40 polyadenylation sequence. Usually, the polyadenylation signal is located 3′ to the recombinase.

i. Recombinase

Generally speaking, a recombinase may be a tyrosine recombinase (i.e. a recombinase that uses a tyrosine-mediated mechanism of recombination) or a serine recombinase (i.e. a recombinase that uses a serine-mediated mechanism). Tyrosine recombinases are well known in the art, and include Cre and FLP. Serine recombinases are also well known in the art, and include phage integrases, such as the [symbol] C31 integrase. In one embodiment, the recombinase is a φC31 integrase. For instance, the site may be an attB site. Nucleic acid sequences encoding a φC31 integrase are known in the art.

ii. Second Vector Promoter

Generally speaking, the nucleic acid of the recombinase is operably linked to a promoter. The terms promoter and operably linked are defined above. Non-limiting representative examples of promoters may include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter. Additionally, the promoter may be a CMV immediate early promoter/enhancer (PCMV) or the CMV enhancer/chicken β-actin promoter (pCAG).

Generally speaking, the promoter should be selected based on the strength of the promoter, the temporal control of the promoter, and the spatial control of the promoter. In some embodiments, the promoter is organ specific. In other embodiments, the promoter is tissue specific. In some embodiments, the promoter is cell specific. For instance, the promoter may be specific for supporting cells in the inner ear. Non-limiting examples of a supporting cell specific promoter are the PLP promoter and the Gfap promoter.

### (c) Composition

The composition of the invention comprises at least one first vector and at least one second vector. The ratio of first vector to second vector may be determined by the use of the composition, for instance, the cell type contacted with the composition, the recombinase used, and the stem cell specific gene used. In some embodiments, the ratio of first vector to second vector maybe 1:1, 1:2, 1:3, 1:4, 1:5, 5:1, 4:1, 3:1, or 2:1.

### III. Cell Comprising at Least One Chromosomally Integrated Stem Cell Specific Gene

Another aspect of the invention encompasses a cell comprising at least one chromosomally integrated stem cell specific gene. Generally such a cell comprises at least one first vector and at least one second vector as detailed in Section II above. Usually, within such a cell, the recombinase of the second vector is expressed. The recombinase recognizes the recombinase site of the first vector and a recombinase site within the chromosomal DNA of the cell, and consequently integrates the stem cell specific gene into the chromosomal DNA of the cell. In some embodiments, the integration event alters the recombination sites such that the integration event is non-reversible.

The recombinase site within the DNA of the cell will typically correlate to the recombinase site of the first vector. Stated another way, the same recombinase will generally recognize the recombinase site of the first vector and the recombinase site within the DNA of the cell. For instance, if the recombinase site of the first vector is a LoxP site, then the recombinase site within the DNA of the cell will be a LoxP site. Similarly, if the recombinase site of the first vector is an attB site, the recombinase site within the DNA of the cell will be an attP or a pseudo-attP site. In some embodiments, the cell is engineered to comprise a recombination site within the DNA of the cell. Methods of engineering a cell to comprise a recombination site are well known in the art. In other embodiments, the cell naturally comprises recombination sites (i.e. no engineering is required for the cell to comprise the recombination site within the DNA of the cell).

Suitable cells may include cells from an organism that expresses stem cell specific genes. Non-limiting examples may include cells from laboratory animals and experimental models, non-human primates, and humans. For instance, non-limiting examples of laboratory animals and/or experimental models may include rodents, such as mice, rats, and guinea pigs, dogs, Drosophila, and Caenorhabditis elegans. In one embodiment, the cell is a skin cell. As used herein, the term “skin cell” refers to a cell derived from the skin of a subject. Skin cells may encompass skin stem cells. Alternatively, the cell may be a sensory hair root cell. In another alternative, a cell from the olfactory epithelium may be used.

Methods to make a cell comprising at least one chromosomally integrated stem cell are well known in the art. Similarly, methods of making a cell comprising at least one first vector and at least one second vector are well known in the art. Such methods may include transfection or transformation techniques such as electroporation, heat shock, calcium phosphate, magnetofection, dendrimers, lipofection, lipid-cation based transfection, transfection via gene gun, and transfection using viral-based vectors. Additionally, commercially available transfection reagents may be used, such as Lipofectamine, Fugene, jetPEI, or DreamFect. Suitable viral vectors may include retroviruses, adenovirus based vectors, herpesvirus based vectors, adeno-associated viruses, vaccinia virus, foamyvirus, lentivirus, and poxvirus vectors. Such methods may be applied to cells in vitro, ex vivo, in vivo, or in situ.

In one embodiment, the cell is contacted with the composition comprising at least one first vector and at least one second vector using lipofection. In another embodiment, the cell is contacted with the composition using liposomes. In yet another embodiment, the cell is contacted with a composition comprising at least one first vector, at least one second vector, N-[1-(2,3-Dioleoloxy)propyl]N,N,N-trimethylammonium methylsulfate (DOTAP), and cholesterol.

IV. Ear Cell Generated from a Skin Cell Derived Stem Cell

In yet another alternative, the cell is an ear cell generated from a skin cell derived stem cell. As used herein, the term “ear cell” refers to a cell that may be found in the ear of a subject. In some embodiments, the term ear cell refers to a cell that may be found in the inner ear of a subject. Non-limiting examples of ear cells may include sensory neurons, hair cells, supporting cells, and non-sensory epithelial cells.

In some embodiments, an ear cell may be generated from a skin cell derived stem cell by increasing the activity of Atoh1 in a skin cell derived stem cell.

### V. Methods for Generating a Skin Cell Derived Stem Cell

Yet another aspect of the invention encompasses methods for generating a skin cell derived stem cell. The method comprises, in part, integrating at least one stem cell specific gene into the chromosomal DNA of a skin cell, wherein the stem cell specific gene is operably linked to a promoter, wherein the promoter's activity is sensitive to the concentration of an activator, and expressing the stem cell specific gene to generate a skin cell derived stem cell.

In some embodiments, the stem cell specific gene is expressed in vitro, ex vivo, in vivo, or in situ.

The method for expressing a stem cell specific gene typically comprises contacting a cell with a composition comprising at least one first vector and at least one second vector of the invention as described above. Generally speaking, after contacting the cell with the composition, the cell comprises at least one first vector and at least one second vector. Usually, within such a cell, the recombinase of the second vector is expressed. The recombinase recognizes the recombinase site of the first vector and a recombinase site within the DNA of the cell, and consequently integrates the stem cell specific gene into the DNA of the cell. In some embodiments, the integration event alters the recombination sites such that the integration event is non-reversible. Generally, the integrated nucleic acid sequence of the stem cell specific gene is transcribed and translated such that the stem cell specific gene is expressed by the cell.

Methods of contacting the cell with the composition are known in the art. Such methods may include transfection or transformation techniques such as electroporation, heat shock, calcium phosphate, magnetofection, dendrimers, lipofection, lipid-cation based transfection, transfection via gene gun, and transfection using viral-based vectors. Additionally, commercially available transfection reagents may be used, such as Lipofectamine, Fugene, jetPEI, or DreamFect. Suitable viral vectors may include retroviruses, adenovirus based vectors, herpesvirus based vectors, adeno-associated viruses, vaccinia virus, foamyvirus, lentivirus, and poxvirus vectors. In one embodiment, the cell is contacted with the composition using lipofection. In another embodiment, the cell is contacted with the composition using liposomes. In yet another embodiment, the cell is contacted with a composition comprising at least one first vector, at least one second vector, N-[1-(2,3-Dioleoloxy)propyl]N,N,N-trimethylammonium methylsulfate (DOTAP), and cholesterol.

In some embodiments, at least one stem cell specific gene is expressed. In other embodiments, at least two, at least three, at least four, or at least five stem cell specific genes are expressed. Suitable examples of stem cell specific genes may include Oct4 (also known as Pou5f1), Nanog, Sox2, GATA3, Neurog1, KLF4, c-MYC, and LIN28.

A recombinase may be used to integrate at least one stem cell specific gene into the chromosomal DNA of the cell. Suitable recombinases are detailed above.

VI. Methods for Generating an Ear Cell from a Skin Cell Derived Stem Cell

Yet another aspect of the invention encompasses methods for generating an ear cell from a skin cell derived stem cell. The methods comprise, in part, inducing the skin cell derived stem cell to differentiate into an ear cell. Methods of differentiating stem cells into ear cells are known in the art. In one embodiment, the activity of Atoh1 is increased in the skin derived stem cells.

VII. Methods of Generating an Ear Cell from a Skin Cell

Another aspect of the invention is a method for generating an ear cell from a skin cell. Generally speaking, the method comprises integrating at least one stem cell specific gene into the chromosomal DNA of the skin cell, wherein the stem cell specific gene is operably linked to a promoter, wherein the promoter's activity is sensitive to the concentration of an activator; expressing the stem cell specific gene to generate a skin cell derived stem cell; and inducing the skin cell derived stem cell to differentiate into an ear cell.

In some embodiments, the method comprises integrating at least one stem cell specific gene into the chromosomal DNA of the skin cell, wherein the stem cell specific gene is operably linked to a promoter, wherein the promoter's activity is sensitive to the concentration of an activator; administering an activator to induce expression of the stem cell specific gene thereby generating a skin cell derived stem cell; stopping the administration of the activator, and inducing the skin cell derived stem cell to differentiate into an ear cell.

### VIII. Methods of Decreasing Hearing Loss

The invention further encompasses methods of decreasing hearing loss. In one embodiment, the method comprises administering a skin cell derived stem cell to the subject. In another embodiment, the method comprises administering an ear cell generated from a skin cell derived stem cell to a subject.

Methods of administering a skin cell derived stem cell, or an ear cell generated from a skin cell derived stem cell, to a subject are known in the art. Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally or intrathecally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

For therapeutic purposes, formulations for administration of the composition may be in the form of aqueous or non-aqueous isotonic sterile injection solutions or suspensions. The composition may be comprise water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Methods of administration may also include infusion with an osmotic minipump, direct microinjection into the cochlea, and application of the composition to the round window membrane.

The decreased hearing loss may be congenital, or it may be acquired. The hearing loss may have been caused by loud noise, aging, infections, and ototoxic chemicals, among which are aminoglycoside antibiotics and platinum-containing antineoplastic agents such as cisplatin.

In some embodiments of the method, the cells are administered before substantial hair cell loss or destruction in at least one organ of Corti in a subject. In this context, substantial means at least 60%, at least 70%, at least 80% or at least 90% loss of or destruction of hair cells in at least one organ of Corti. In other embodiments, the composition is administered during hair cell loss. In still other embodiments, the composition is administered after substantial hair cell loss. In further embodiments, the composition may be administered before, during, or after a cochlear implant is inserted. Methods of inserting a cochlear implant are known in the art.

Suitable subjects may include subjects that comprise an organ of Corti. For instance, non-limiting examples may include laboratory animals, non-human primates, and humans. Non-limiting examples of laboratory animals and/or experimental models include rodents, such as mice, rats, and guinea pigs, dogs.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Examples 1-5

### Introduction

Development of the vertebrate ear is a coordinated molecular transformation of a set of epidermal cells (the otic placode) into the fully developed ear with its neurosensory component, necessary for signal extraction and transmission, and the nonsensory component, forming the labyrinth necessary for directing sensory stimuli to specific sensory epithelia (FIG. 1). Three developmental steps ensure that (1) the ectoderm is transformed to otic ectoderm, including neurosensory precursor cells (2), neurosensory precursor cells generate neurons, and (3) sensor precursor cells form hair cells and supporting cells in the designated area of sensory epithelia (FIG. 1). As with other developing systems, differentiation of the epidermal cells into the four major cell types of the ear (sensory neurons, hair cells, supporting cells and non-sensory epithelial cells) occurs through molecular fate specification followed by clonal expansion of committed precursors to produce the final number of a specific cell type in embryos. These neurosensory cells have a limited life span that is further truncated by numerous environmental insults (loud sound, ototoxic substances such as cysplatin or aminoglycoside antibiotics) and genetic predisposition (numerous genes related to hearing loss). Combined with the increased longevity of humans, genetic predisposition and cumulative insults lead to an increasing likelihood of neurosensory hearing loss with age, thus depriving half of people age 70 and older from one of the most important aspect of communication as well as negatively affecting their sense of balance.

Much like with the adult human brain (1), there is only limited evidence for the presence of neurosensory stem cells in the mammalian ear that seem to be able to proliferate only under certain circumstances in vitro (2,3). Consequently, loss of any differentiated neurosensory cell will potentially diminish hearing. In contrast to other vertebrates (like bony fish or chickens), there is no evidence for spontaneous regeneration of lost neurosensory cells in the mammalian cochlea in vivo. Because of the difficulties in accessing these stem cells in the adult human ear without disrupting the very organ that requires regeneration, other sources of stem cells and strategies are being explored that may ultimately provide replacements for lost neurosensory cells or restore hearing:

An already existing therapy is to use remaining sensory neurons in combination with a cochlear implant (an electric device that transforms sound into electric stimuli) to bypass the missing hair cells by directly stimulating nerve fibers, bringing sound information via the sensory neurons to the brain. The viability of this approach rests on the long-term survival of sensory neurons that depend on neurotrophic support from the lost hair cells and the dedifferentiating supporting cells for their survival (4,5). To maximize the viability of sensory neurons, several strategies are being explored using neurotrophin infusions (6-8). Attempts are being made to understand the molecular mechanism that shuts down neurosensory proliferation in the ear through regulation of cyclin-dependent kinase inhibitor expression (9) in analogy to other systems (10). Conceptually, it seems possible to translate those insights directly into reactivation of the dormant replacement capacity of mammals, comparable to the injury-induced regeneration of chicken hair cells. Recent work has demonstrated that cell cycle reentry is possible in neonatal mammals (11) but manipulation of this pathway is not without risks (9,12) requiring a more sophisticated manipulation of this pathway than simply knocking out cyclin-dependent kinase inhibitor genes.

Proliferation of postmitotic neurosensory cells can be forced through targeted deletion of S-phase entry control genes such as the retinoblastoma gene (13,14). While such approaches lead to the transient formation of more hair cells and can potentially be initiated via siRNA therapy, such cells ultimately die necessitating further refinement of this approach before it can be therapeutically useful. Transdifferentiation of the supporting cells of the sensory epithelium into hair cells can be enforced through overexpression of regulatory genes (15). The problem with such a gene therapy approach is that it will deplete the existing supporting cells, thus leaving the sensory epithelia in an unusual organization with limited functionality of the organ of Corti which, in part, depends on supporting cells (16). Stem cells of various tissues are being investigated and some have been successfully incorporated into the developing chicken ear, providing proof of principle for a stem cell approach (17,18). However, only a limited set of stem cell sources have been investigated. Thus far, the easily accessible stem cells derived from hair follicles (19-22) have not been explored for ear regeneration.

The purpose of the following examples is to analyze molecular steps that specify the cell fate of neurosensory hair cells out of epidermal cells and that regulate the clonal expansion of those precursors and their differentiation into sensory neurons and hair cells. After presenting these developmental steps, the potential use of skin-derived stem cells to generate neurosensory precursors useful for ear implantation will be discussed.

### Example 1

### Turning Embryonic Ectoderm Cells into Otic Neurosensory Cells: the Molecular Basis for Otic Neurosensory Induction

Induction of the ear requires both mesodermal and neuroectodermal signals (23). This basic decision is essentially identical to the induction of the neural plate (24,25) and olfactory system (26). Similarly to these neural inductions, ear induction is based on FGFR signaling, possibly combined with inhibition of BMP signaling (FIG. 1a). Molecularly, these inductions require diffusible signals that cause graded responses in the target cells. Four such diffusible signals have been characterized in mammalian ear development: SHH from the floor plate and notochord (27), FGF8, FGF10 and FGF3 from mesoderm and neuroectoderm (28), WNTs from the hindbrain (29,30) and BMP4 from general ectoderm as well as from the ear (31). The combined action of these signals change the fate of ectodermal cells to acquire an otic placode phenotype instead (FIG. 1a). Within the otic placode, the acquisition of a neurosensory phenotype is consolidated with the upregulation of the proneuronal gene neurogenin 1 (Neurog1). Upregulation of Neurog1 was detected as early as E8.75 in the mouse in a few cells (32) and is thus not unlike the sensory organ precursor cell known to initiate formation of mechanosensors in insects (33-36). In contrast to most insect mechanosensory organs, the mammalian ear undergoes many more cell cycles to expand first the precursor population followed by a coordinated cell cycle exit of, in order, sensory neurons, hair cells and supporting cells (37). The adult mouse ear contains approximately 10,000 hair cells and 11,000 sensory neurons. Between embryonic day 8.75 (first expression of the bHLH gene Neurog1) and E13.75 (when all cochlear and most vestibular hair cells and neurons have exited the cell cycle) ear precursors will undergo approximately 16 cell cycles of about 8.5 hours each (37). Assuming only symmetric divisions, only two initial cells would be needed to generate 32,000 neurosensory cells of the adult mouse ear in only 15 rounds of division. Selecting the right number of cells that express Neurog1 is therefore a crucial final step of otic placode induction.

Neurog1 is not only one of the earliest genes to identify cells of the otic placode but it also has an essential functional role in ear development: Neurog1 is necessary for all sensory neuron formation (32). However, Neurog1 also affects other aspects of ear development, including development of sensory epithelia and hair cells (38,39). Misexpression of Neurog1 in frog skin demonstrates that it is not only necessary but also sufficient to induce neuronal transformation of epithelial cells (40). Understanding otic induction requires therefore a mechanistic understanding of how the four above outlined diffusible factors (SHH, WNTs, BMPs and FGFs) interact at a cellular level to change ectodermal cells to otic cells and eventually to a neurosensory precursor fate by upregulating Neurog1. The ubiquitous use of these factors in neuronal and non-neuronal systems alike suggests that they are necessary but not sufficient to achieve this epithelial transformation. Other transcription factors possibly important for the epithelial-neurosensory transition are also early expressed in the placode such as Gata3 (41), Pax2/8 (42,43), Tbx1 (44), Foxg1 (45,46), Foxi1 (47), Eya1/Six1 (48) and Oct4 (49). In particular, the unique overlapping expression of Pax2/8, Gata3, Foxg1, Foxi and Eya1/Six1 may provide a necessary context for inner ear neurosensory development that is dramatically altered in their absence. How do all these factors interact with each other to achieve epithelial-to-otic transformation? A central cellular event in many cells to induce cell fate changes is regulation of transcription factors via modifying BMP signaling. BMPs signal through dimerized BMP receptors to phosphorylated SMADs (50) that then enter the nucleus to regulate over 500 genes. Entry of SMADs to the nucleus and binding to promoters is tightly regulated by numerous interactions with other signaling pathways, notably the FGF- and EGF-related receptor tyrosine kinase (RTK) signaling pathways (FIG. 1). Activation of the RTK pathway will block SMAD entry to the nucleus (50). GATA3 can form complexes with SMADS and thereby change binding specificity (51). Combined with its role in hair follicle stem cells (20), the early expression and massive reduction of ear development in Gata3 null mice (41) shows that this gene plays an important role in setting up the proliferation capacity of the otocyst through interactions with SMADs (51) and FGFs (52). Some evidence for PAX signaling affecting SMADS exists for thyroid development (53), but this has not been demonstrated for the ear. However, an absence of sensory neurons has been claimed for Pax2 null mice (42), a claim that needs to be reexamined with more sophisticated techniques. FOXG1 has recently been shown to interfere with the SMAD-FOXO complex and thus can alter SMAD-mediated gene regulation (54) and neurosensory development is altered in Foxg1 null mice (46). WNT signaling through β-catenin is known to act directly on SMAD-mediated gene activation (50), but other interactions of WNTs and BMPs are known and which of these pathways is active in the ear requires further research. Wnt signaling clearly effects otic placode formation (30) and later ear development (29), but the effects of β-catenin on SMAD signaling have not been investigated in the ear. Oct4 null zebrafish show no expression of Neurog1 in the otic placode, suggesting that OCT4 regulates Neurog1 (49). Such an epistatic effect of Oct4 has recently been demonstrated in mammalian stem cells (55) but has net yet been shown for the mammalian ear. In the brain, NEUROG1 inhibits SMAD1-mediated signaling by sequestering the SMAD1 complex away from glia-specific promoters, thereby enhancing a neuronal phenotype (50). Thus, NEUROG1, once expressed, could further downregulate SMAD signaling in the otic placode, enhancing the commitment toward neuronal development. Tbx1 is known to suppress unmitigated SMAD1 signaling, thereby converting neurosensory fate back to epithelial fate. Finally, SHH is known to upregulate bHLH genes in somites and there is neither Neurog1 upregulation nor sensory neuron formation in Shh null mice (27). Thus, SHH could affect SMAD1 phosphorylation indirectly through expression of Neurog1, possibly allowing Neurog1 expression only in cells with a specific concentration of BMP and SHH signaling, like in the spinal cord (56). Indeed, recent in vitro data on embryonic stem cells how that treatment with SHH can bias toward hair cell differentiation, albeit at a very low yield (57). Whether SHH's effects in vitro are accomplished via regulation of SMAD signaling through Neurog1 expression requires further research. In addition, for a therapeutically useful yield of cells, the propensity for neurosensory differentiation must be increased.

Taken together, these data suggest that several otic transcription factors expressed early in development and diffusible morphogens co-operate to modify BMP-SMAD signaling thereby altering epithelial fate toward neurosensory otic placode fate. While SMADs undoubtedly play a role in ear development, exactly when and where Smad's are expressed and phosphorylated in mammalian ear development requires further analysis. Presently we only know that, in zebrafish, Smad1 is expressed in the sensory neurons of the ear (58) consistent with our hypothesis that SMAD regulation by various means may be a crucial first step in ectodermal-otic transition. It needs to be noted that most of the molecules thus far identified are used in many other developing systems, suggesting that specific otic identification is achieved through a unique combination of genes and not through a single gene unique to the otic placode. Independent of this uncertainty, the final step in otic neurosensory commitment is the upregulation of Neurog1, consolidating the switch from epidermal to proneurosensory fate and initiating pro-neurosensory clonal expansion. Therefore, the molecular basis that makes this clonal expansion possible and turns a small set of otic placode cells into the several thousand neurosensory cells of the adult ear is discussed below.

### Example 2

### The Molecular Basis of Inner Ear Neurosensory Cell Generation

While the presence of Neurog1-expressing precursors is obvious at E8.75 (32), neither the entire fate of these proneuronal precursors nor the distribution of prosensory precursors is fully known. The first identification of sensory patches that will give rise to hair cells and supporting cells is only possible around E10.5. At this stage or later, several genes highlight to various degrees those prosensory areas, notably the neurotrophins BDNF and Ntf3 (59,60), Bmp4 and Lnfg (31), Sox2 (61), Islet1(62) and Fgf10 (63). Several of these genes are expressed both in the otocyst wall in likely sensory epithelial precursors as well as in delaminated, proliferating neuronal precursors (59,62,63), suggesting a possible common precursor for both sensory epithelia and neurons.

This common expression in the otocyst wall and delaminated neuronal precursors is also true for Neurod1 (64,65), a bHLH gene that is regulated in the ear by Neurog1 (32). However, whereas Neurog1 null mice have a severe reduction in hair cells, notably in the saccule and cochlea (38), there is only a limited shortening of the cochlea in Neurod1 null mice (66). This suggests that some precursors that express Neurog1 are also forming hair cells and supporting cells of sensory epithelia, whereas precursors that express Neurod1 are already committed to the neuronal lineage. Recently, it was shown that some sensory precursors switch their fate in the absence of Neurog1 and differentiate into hair cells (39). In addition, using sensitive markers, it was shown that some sensory neurons express the otherwise hair-cell-specific bHLH gene Atoh1, a gene essential for hair cell differentiation (67,68). These indirect suggestions for a clonal relationship between some sensory neurons and hair cells was confirmed with lineage tracing in chicken (69). Combined, these data suggest that at E10.5 the neurosensory precursors may be composed of three populations: (1) neuronal precursors that form only neurons, (2) neurosensory precursors that form only hair cells (and supporting cells) and (3) precursors that form both neurons and hair cells (FIG. 2). How the selection of these precursors and the determination of their relative size are regulated and whether or not there is a coordinated transition of one precursor into another as in brain development (70) remains unclear. But the existence of a population that can generate both hair cells and neurons from a single line of clonally related cells has therapeutic potential: it would allow for the transformation of neuronal stem cells that give rise to both neurons and hair cells out of the same stem cell. Indeed, recent in vitro data suggest that the yield of hair cells out of bone marrow stem cells can be enhanced when stem cells are selected that express neuronal markers before they are switched to a hair cell differentiation pathway (Heller et al, unpublished data). Still, the question remains: what is the function of two or more, instead of one bHLH gene in the neuronal development of the ear? Our understanding of the development of the olfactory system provides clues to begin to answer this question. In the olfactory system, transient amplifying precursors are initially specified by Mash1. The Mash1-expressing precursor gives rise to a transient amplifying precursor population, the immediate neuronal precursor (INP), which expresses Neurog1. INP cells divide, exit the cell cycle accompanied by Neurod1 expression and differentiate into olfactory receptor neurons (71,72). Both Fgfs and Bmps play a role in specifying the transition from one cell type to the next and hence the degree of clonal expansion (73,74) and allocation to various clones giving rise to olfactory neurons and cells of the olfactory system (72). As in muscle cell proliferation, an antagonistic interaction between GDF11 and follistatin determine the expression level of the cyclin-dependent kinase inhibitor 1b (Cdkn1b; formerly p27 kip) and thus determine the cell cycle exit (75). Comparable to the olfactory system, the ear shows various progenitor populations able to produce either hair cells, supporting cells, and even sensory neurons or hair cells and supporting cells (69). Cell cycle exit in these progenitors is regulated by cyclin-dependent kinase inhibitors (9). However, the regulation of the cyclin-dependent kinase inhibitors by GDF 11/follistatin remains to be shown for the ear. Nevertheless, it appears that, in neurosensory development of the ear and olfactory epithelium, we can distinguish a phase of early clonal expansion with limited, if any expression of cyclin dependent kinase inhibitors followed by a phase of progressive upregulation of these inhibitors to tightly regulate the final number of neurosensory cells (9,11). The molecular basis of this final phase of progenitor cell cycle regulation and differentiation into distinct cell types is well understood in the ear (11,13,39,76,77). Therefore, the next example concentrates on the molecular basis of clonal expansion of neurosensory precursors to provide the right number that can then be regulated to divide and terminally differentiate through these molecularly known pathways.

### Example 3

### Molecular Basis of Otic Neuronal Stem Cell Maintenance and Expansion

Recent years have revealed the molecular basis of stem cells in general, which involves the genes Oct4, Nanog and Sox2 (55), and of neuronal stem cells in particular, involving certain bHLH genes (78). Not surprisingly, WNT and SHH signals seem to interact with bHLH genes to ensure clonal expansion of neuronal stem cells (79). Not all the details are clear yet for the ear, but several important aspects are known that suggest a rough parallelism to this general principle with ear-specific molecular players. SHH and WNT1/3A are diffusible signals that influence ear histogenesis and morphogenesis from sources outside the ear (27,29,30). In addition, FGF's likely signaling through FGFR2B (80) affect morphogenesis and neurosensory formation (52,63,81,82). How signals generated by these diffusible factors combine with local signals such as EYA1 (48) to maintain and alter bHLH-gene-mediated neuronal progenitor specification and proliferation is unclear. Based on the limited data and expanding general principles validated in other systems, the following tentative conclusions can be drawn: in general, neuronal stem cells express both glial and neuronal markers such as GFAP and Nestin (79) but also the activator and repressor-type bHLH genes (78). Eliminating the repressor-type bHLH gene signaling initiates premature neuronal differentiation combined with limited clonal expansion (78). This can either be achieved by eliminating Hes genes, Notch genes or the intracellular partners that regulate Hes expression (RBPSUH, formerly RBP-J), or by changing the ability of HES to form homodimers that bind to N-boxes using the WRPW domain (FIGS. 1,3). An excellent example of the latter is the reduced clonal expansion and premature neuronal differentiation in the forebrain of Foxg1 null mice (83), in part mediated by alteration in DNA binding of HES homodimers interacting with TLE and RUNX (84). Neurog1 drives the upregulation of several genes relevant for the maintenance of neuronal stem cells. The expression of the NOTCH ligand DELTA 1 is delayed in Neurog1 null mice, showing that Neurog1 is epistatic to DELTA 1 (32). Consistent with other developing mammalian neuronal systems (78), initial upregulation of Neurog1 is not ubiquitous but occurs in a few cells only. Nevertheless, eliminating RBPSUH and thus the NOTCH signaling pathway (FIGS. 1,3) results in expansion of Neurog1-expressing areas of the ear (32). These data show that NEUROG1 signaling affects Notch signaling and may indeed be effective at this early time. Despite the known presence of Notch and several ligands as early as E8.5 (85,86) and the known effects of deletions of Notch ligands on ear development (87-89), there is no direct evidence suggesting expression of any Hes genes in the ear prior to E12.5 (85). Given that activated NOTCH signals through de-repression of Rbpsuh and thus upregulation of Hes1 and Hes5, the expression data are bound to be incomplete and further studies using more sensitive techniques such as green-fluorescent-protein-expressing reporter systems (90) are needed to reveal the spatial and temporal pattern of Hes distribution in the developing otocyst. Thus, at the moment, the role of Hes signaling in neuronal and early neurosensory stem cells of the ear remains unclear (FIG. 1).

Altering the balance between Hes and activator-type bHLH genes determines how long a stem cell cycles and whether they differentiate toward a neuronal or a glial cell type (78). Eliminating all activator-type bHLH genes can result in phenotypic switch to a glial phenotype (72). Such switches in phenotype combined with truncation of later formed cells such as hair cells or supporting cells have been described in Neurog1 null mice (38,39). Most interestingly, Neurod1, a bHLH gene that is immediately downstream of Neurog1 and depends on Neurog1 for early expression (32), shows a profound upregulation in hair cells that exit the cell cycle prematurely in Neurog1 null mice (39). Likewise, altering NOTCH signaling, either at the level of ligand/receptor (87,88), the intracellular effectors Hes1 and Hes5 (91), or a co-factor for binding to the N-box (46), results in aberrations of hair cell organization. Combined, these data show that proper bHLH signaling is essential for normal neurosensory development of the ear and requires the interaction of both activator and inhibitor-type bHLH genes for transit amplification of precursors. The ear is in this respect essentially identical to other developing neuronal systems (72,78,79), although it uses a unique combination of players.

### Example 4

### Forming the Right Number of Hair Cells

### Complex Regulation of a Simple Outcome

In addition to the above-outlined molecular interactions that result in the formation of sensory neurons and neurosensory precursors, a partially overlapping set of genes regulates the neurosensory and supporting cellular components of the inner ear sensory epithelia development (FIG. 1c). These regulations involve the bHLH network of the neuronal activator gene Atoh1 (39,67), and the repressor genes, Hes1 and Hes5 (91,92), in combination with Notch1, and the delta and jagged/serrate ligands, DII1, Jag1 and Jag2 (85,88). These two networks are directly linked (FIG. 3) through the expression regulation of and interactions with the Hes genes (78). The bHLH network functions through the DNA targeting and binding affinities of a combinatorial complex of proteins (78) that involve bHLH dimmers (93), transducin-like enhancer of split (Tle, groucho in fly), runt-related transcription factor (Runx), and forkhead box G1 protein (Foxg1) (84,94-96). The TLE protein is the central component with binding sites for HES, runt and forkhead proteins and forms the repressor complex that, in general, prevents neurogenesis (FIGS. 1,3). HESs also exert an additional effect by competing with the activator bHLH proteins for the ubiquitously expressed class I bHLH activator binding partner (E protein), Tcfe2a (FIGS. 1,3). TCFE2A functions by facilitating the formation of heterodimers with activator-type bHLH genes (NEUROG1, ATOH1 and certain HESs) that permit binding to the E-box (5-CANNTG-3). Homodimers of activator bHLH proteins either have low E-box-binding affinities or are inactive (97). HES homodimers bind N-box response elements (5-CCGGAA-3). HES-mediated repression is largely through the Orange and WRPW protein domains. The Orange domain confers specificity for homodimerization among the HES family members and the WRPW domain interacts with the co-repressor TLE protein for enhanced binding to N-boxes. A second class of repressor bHLHs are represented by the inhibitor of DNA-binding (Id) bHLH genes that function as a dominant negative protein due to the absence of the DNA-binding motif (77). Strength of activation or repression can be further fine-tuned by qualitative and quantitative ratios of these proteins and paralogue usage (98-100). HES6 differs in that it can function as a positive-feedback loop in neurogenesis by forming heterodimers with other HESs, inhibiting their repressor activity (101-103).

This intracellular signaling network is tied into an intercellular signaling network that refines fate assignment of hair cells and supporting cells in the sensory epithelia through NOTCH signaling (FIGS. 1,3). NOTCH signaling contributes to proliferation, apoptosis, stem cell self-renewal and regulation via lateral inhibition between neighboring cells (85,104). In vertebrates, Notch receptors all share similar functional domains, where the extracellular domain has epidermal growth factor and Lin-Notch repeats (LNR) and the intracellular domain has a RBPSUH-associated motif (RAM). Homomerical oligomerization of the NOTCH receptors and subsequent differential proteolytic cleavage of the intracellular domain (ICD) are modulated by two classes of ligands that induce (Serrate/Jagged) or inhibit (Delta) signaling. The presence of extracellular Fringe modifies NOTCH to signal only with Delta proteins, whereas unmodified NOTCH is responsive to Jagged (105). Upon binding a ligand, intracellular cleavage by a variable γ-secretase complex containing presinilin related molecules leads to a NOTCH fragment that interacts with RBPSUH to regulate Hes expression (FIGS. 1,3).

Examination of the inner ear phenotype of mutants for many of these pathway component genes reveals several levels of severity. The least severe are those that alter the cell numbers and rows in the organ of Corti. These include Cdkn1a (formerly p21), Cdkn2d (formerly p19Ink4d), Hes6, Hes1, Hes5, Notch1, and Jag2 (9,86,91,101,106,107) with more severe changes in the organ of Corti being observed in the Neurog1, Foxg1, Jag1 and Cdkn1b (formerly p27) mutants (38,39,46,87,108). In contrast to the limited addition of hair cells in Cdkn null mice (9,11,106), conditional null of the Rb1 gene causes a preferential expansion of the hair cell population leading to cochlear tumors (13,14).

Beyond these readily understandable effects on inner ear differentiation are less obvious effects that require a deeper insight into the molecular interactions to appreciate them. Some of these effects require the additional interaction of activator-type bHLH genes, more specifically of Neurog1 and Atoh1. In Atoh1-deficient mice, only the differentiation of hair cells is affected with no effect on morphogenesis or formation of undifferentiated precursors in specific sensory epithelia (FIG. 4). In contrast, in the Neurog1 null mice, all inner ear ganglion neurons are absent (13,39) and there are morphogenetic effects such as a reduction of hair cells by 40-80%, depending on the sensory epithelium (FIG. 4). This suggests that the proliferative capacity of neurosensory precursors is also being affected in these activator bHLH-deficient mice. Recently, an interactive network of activator- and inhibitor-type bHLH genes has been described that tightly regulates the proliferation and differentiation of retinal ganglion cells (109). Specifically, this interaction is mediated with paralogs of two inner ear bHLH genes, Neurog2 and Atoh7 (formerly Math5). It appears that Atoh7 is more profoundly affected by high levels of Hes, possibly through an inhibitory action of Hes homodimers on N-boxes in its promoter region (FIG. 5). In contrast, Neurog2 is compatible with high levels of Hes and promotes continuous cycling of the precursors. Through as yet unclear extracellular signals, possibly mediated by the Delta-Notch system, Hes expression is downregulated, thereby decreasing inhibition of Atoh7 expression. Once ATOH7 protein has reached a critical level, most E proteins will form heterodimers with ATOH7, reducing NEUROG2/E-protein heterodimer signaling. These phases were shown to neatly correlate with clonal expansion (high levels of Neurog2 and Hes), cell cycle exit (equal level of Neurog2 and Atoh7, reduction in Hes) and differentiation (reduced presence of Hes and Neurog2, high expression of Atoh7) of retinal ganglion cells (FIG. 5). While impressive in the technical achievements of single cell quantitative PCR, even this work leaves open the questions open of protein-protein interactions and the half-life of bHLH proteins. Nevertheless, it stresses that technical advances are needed to close the gap between the most-sensitive tissue based detection systems and the more-sensitive non-tissue based detection.

A similar regulation is conceivable in the ear, involving instead Aoth1 and Neurog1 and may also play a role in neuronal differentiation of the ear (Neurog1 and Neurod1) and the olfactory system (Mash1, Neurog1, Neurod1). In this context, it is important not only that Neurog1 absence has been shown to reduce formation of hair cells and also to result in loss of sensory neurons, but also that Atoh1 upregulation was recently shown much earlier in the ear using more sensitive detection systems and some sensory neurons were found to express Atoh1 (39). These data support the idea that at least some hair cells are clonally related to sensory neurons and this precursor population may be larger in the mammalian ear compared to the limited clonal relationship thus far found in chicken development (69). Consistent with the comparatively late upregulation of CDK inhibitors in the ear (9,106), these data suggest that the initial clonal expansion of neurosensory precursors in the ear may be predominantly regulated via bHLH gene interactions and their effect on cell cycle progression with only limited input from the Delta-Notch system (FIGS. 3,5). How genes that define sensory epithlia and may be upstream to Atoh1 regulation such as Sox2 (61) affect this intracellular signaling remains at the moment unclear as Sox2 might not be the only factor driving upregulation of Atoh1. In summary, these data show a complex intracellular signaling for neurosensory precursor regulation that requires transition between several activator-type bHLH genes that provide the molecular basis for transit-amplification and precursor specification. Some extracellular signals that regulate the expression of activator-type bHLH genes are known in the ear (Sox2, Oct4, Tbx1) and the role of the Delta/Notch regulation in refining hair cell and supporting cell development is becoming clear. How regulation of Cdk inhibitors ultimately causes the irreversible arrest of cell cycle re-entry remains as unclear in the ear (11) as in other non-proliferating systems (10).

### Example 5

### Using Easily Accessible Skin and Olfactory Precursors to Regenerate Hair Cells of the Ear

In these examples, the current understanding of the molecular basis for ear neurosensory specification and proliferation have been outlined. Clearly, this involves the transformation of ectodermal cells into neurosensory cells through the selective expression of a reasonably well understood sequence of gene activations. Interestingly, several of the genes found to be important in ear neurosensory development are also important for skin stem cells. For example, Gata3 is needed (together with other genes) for hair follicle stem cell determination (20). GATA3 acts with Lef-1/Wnts to define the inner root sheath versus the hair shaft cell fate decision in hair follicle morphogenesis (20). GATA3 is essential for early ear development and is expressed already in the invaginating ectodermal placode (41). Thus, isolating hair follicle precursors from skin would provide progenitor cells that have already one of the crucial genes for ear formation expressed. Expression of other crucial genes such as Neurog1, Foxg1, Foxi1 and Pax2/8 in these cells in tissue culture could transform those cells into ear neurosensory precursors able to differentiate into neurons, as already demonstrated with the ectopic expression of Neurog1 in frog ectoderm (40).

Another source of neural-crest-derived stem cells was recently identified in sensory hair roots (21). These cells have the capacity to express neuronal markers if implanted into the spinal cord (22). It is likely that these cells are related to the neural-crest-derived Merkel cells (110), a population of cells that express two genes essential for hair cell development, Pou4f3(111-114) and Atoh1 (67,115). These cells seem to retain their gene expression profile while proliferating. If so, these cells might readily differentiate into hair cells if implanted into ears; this appears to be possible with other stem-cell-derived precursors that are equally characterized by Atoh1 and Pou4f3 expression (17).

Most importantly in this context, recent work has molecularly characterized the only source of continuously proliferating neuronal stem cells in mammals, the olfactory epithelium (72). This epithelium is surgically easily accessible and some precursors are characterized by the expression of the same bHLH genes known for ear neuronal development, Neurog1 and Neurod1(116) and Foxg1 (46,74). Isolation of Neurog1-positive precursors and forced expression of other ear-related genes such as Gata3 (41), Foxi1, Pax2/8 (43) or Fgf10 (63) might help drive such cells in tissue culture towards ear neurosensory development. Clearly, other genes expressed in both the ear and olfactory epithelium, such as Sox2 or Foxg1, would not redirect the fate of these cells beyond olfactory specification.

These approaches might provide sufficient adult cellular stem cell material to restore lost hair cells and sensory neurons of the ear combined with limited surgical intervention to obtain adult stem cells to repopulate the ear. If these simple approaches have too low a yield of cells with ear-specific gene expression, the known steps of neurosensory development in the ear as outlined above can provide appropriate guidance to achieve this goal through additional manipulations. Such manipulations may include, but are not limited to, selective upregulation of miRNA. miRNAs are generally known to be important in cell fate determination and proliferation regulation (117) through regulation of large sets of target genes. Some miRNAs were recently shown to be selectively expressed in hair cells (118) and may be important in consolidating cell cycle exit and maintaining differentiation of neurosensory aspects of the ear but such functions require ear-specific conditional mutations of enzymes necessary for miRNA processing (119). All the progress towards the molecular basis of ear development during the last five years, combined with recent advances in isolation and molecular manipulation of stem cells from various sources, raises the hope that hearing loss will soon be correctable via stem cell therapy before the baby boom generation will have suffered untreatable neurosensory hearing loss.

### Example 6

### Flow Diagram for the Generation of Adult Stem Cells Out of Skin and Their Use for Cellular Therapy of Neurosensory Hearing Loss

### Background

Cells can be derived from biopsy and will be treated with both phage φ31 integrase and vectors containing the following genes (alone or in various combinations): Oct4 (now Pou5 μl), Nanog, Sox2, GATA3 and Neurog1. Those genes have been identified previously as playing unique roles in maintaining embryonic stem cells and providing the transition to neuronal stem cells (55). In the ear, Neurog1 has been identified to be the earliest definite marker of neurosensory components and such precursors can form both neurons and hair cells (38; 39), the two neuronal components that are defective in neurosensory hearing loss.

Approach

Generation of skin derived stem cells: Integration of those five genes singly or in combination into skin derived cells will be achieved using phage φ31 integrase combined with the above listed genes. Each gene will be individually or in tandem spliced to a bacterial erythromycin sensitive promoter element that will A) allow driving gene expression in tissue culture until stem-cell like characteristics are achieved and B) will lead to gene inactivation as soon as the erythromycin is shut off. In addition, these cells will be transfected with a GFP carrying vector.

Usage of stem cells: Skin cells that have been successfully transformed into stem cells using the above outlined approach will be harvested and injected into the ear of a suitable subject and their integration and differentiation will be monitored using GFP that is independently inserted into the skin derived stem cells.

Outcome: It is anticipated that this cellular approach will provide cells that can in a follow-up treatment using Atoh1 induced to differentiate as hair cells in the ear. Such differentiation of cells into hair cells using Atoh1 is already known in the field (15) and is not part of this application, which focuses on preserving the idea of using skin derived stem cells for treatment of hearing loss (121).

Summary: This outlined approach uses for the first time the combination of phage integrase to insert genes known to be part of the stem cell code for the ear to drive stem cell transformation of skin cells to be subsequently inserted into the ear as a cellular therapy against neurosensory hearing loss.

### REFERENCES FOR EXAMPLES 1-6

1. Rakic P. 2006. Neuroscience. No more cortical neurons for you. Science 313:928-929. 26.
2. Rask-Andersen H, Bostrom M, Gerdin B, Kinnefors A, Nyberg G et al. 2005. Regeneration of human auditory nerve. In vitro/in video demonstration of neural progenitor cells in adult human and guinea pig spiral ganglion. Hear Res 203:180-191.
3. Li H, Liu H, Heller S. 2003. Pluripotent stem cells from the adult mouse inner ear. Nat Med 9:1293-1299.
4. Fritzsch B, Tessarollo L, Coppola E, Reichardt L F. 2004. Neurotrophins in the ear: their roles in sensory neuron survival and fiber guidance. Prog Brain Res 146:265-278.
5. Sugawara M, Corfas G, Liberman M C. 2005. Influence of supporting cells on neuronal degeneration after hair cell loss. J Assoc Res Otolaryngol 6:136-147.
6. Roehm P C, Hansen M R. 2005. Strategies to preserve or regenerate spiral ganglion neurons. Curr Opin Otolaryngol Head Neck Surg 13:294-300.

7. Gillespie L N, Shepherd R K. 2005. Clinical application of neurotrophic factors: the potential for primary auditory neuron protection. Eur J Neurosci 22:2123-2133.
8. Staecker H, Brough D E, Praetorius M, Baker K. 2004. Drug delivery to the inner ear using gene therapy. Otolaryngol Clin North Am 37:1091-1108.
9. Chen P, Zindy F, Abdala C, Liu F, Li X, Roussel M F, Segil N. 2003. Progressive hearing loss in mice lacking the cyclin-dependent kinase inhibitor Ink4d. Nat Cell Biol 5:422-426
10. Molofsky A V, Slutsky S G, Joseph N M, He S, Pardal R et al. 2006. Increasing p16(INK4a) expression decreases forebrain progenitors and neurogenesis during ageing. Nature
11. White P M, Doetzlhofer A, Lee Y S, Groves A K, Segil N. 2006. Mammalian cochlear supporting cells can divide and trans-differentiate into hair cells. Nature 441:984-987.
12. Kanzaki S, Beyer L A, Swiderski D L, Izumikawa M, Stover T et al. 2006. p27(Kip1) deficiency causes organ of Corti pathology and hearing loss. Hear Res 214:28-36.
13. Mantela J, Jiang Z, Ylikoski J, Fritzsch B, Zacksenhaus E, Pirvola U. 2005. The retinoblastoma gene pathway regulates the postmitotic state of hair cells of the mouse inner ear. Development 132:2377-2388.
14. Sage C, Huang M, Vollrath M A, Brown M C, Hinds P W et al. 2006. Essential role of retinoblastoma protein in mammalian hair cell development and hearing. Proc Natl Acad Sci USA 103:7345-7350.
15. Izumikawa M, Minoda R, Kawamoto K, Abrashkin K A, Swiderski D L et al. 2005. Auditory hair cell replacement and hearing improvement by Atoh1 gene therapy in deaf mammals. Nat Med 11:271-276.
16. Colvin J S, Bohne B A, Harding G W, McEwen D G, Ornitz D M. 1996. Skeletal overgrowth and deafness in mice lacking fibroblast growth factor receptor 3. Nat Genet 12:390-397.
17. Rivolta M N, Li H, Heller S. 2006. Generation of inner ear cell types from embryonic stem cells. Methods Mol Biol 330:71-92.
18. Li H, Corrales C E, Edge A, Heller S. 2004. Stem cells as therapy for hearing loss. Trends Mol Med 10:309-315.
19. Rhee H, Polak L, Fuchs E. 2006. Lhx2 maintains stem cell character in hair cells. Science 312:1946-1949.
20. Kaufman C K, Zhou P, Pasolli H A, Rendl M, Bolotin D et al. 2003. GATA-3: an unexpected regulator of cell lineage determination in skin. Genes Dev 17:2108-2122
21. Sieber-Blum M, Grim M, Hu Y F, Szeder V. 2004. Pluripotent neural crest stem cells in the adult hair follicle. Dev Dyn 231:258-269.
22. Sieber-Blum M, Schnell L, Grim M, Hu Y F, Schneider R, Schwab M E. 2006. Characterization of epidermalneural crest stem cell (EPI-NCSC) grafts in the lesioned spinal cord. Mol Cell Neurosci 32: 67-81.
23. Groves A K. The induction of the otic placode. In: Kelley M W, Wu D K, Popper A N, Fay R R, editors. Development of the inner ear. New Springer Verlag; 2005. p10-42.
24. Lemaire P, Bertrand V, Hudson C. 2002. Early steps in the formation of neural tissue in ascidian embryos. Dev Biol 252:151-169.
25. Delaune E, Lemaire P, Kodjabachian L. 2005. Neural induction in Xenopus requires early FGF signalling in addition to BMP inhibition. Development 132:299-310.
26. Shou J, Rim P C, Calof A L. 1999. BMPs inhibit neurogenesis by a mechanism involving degradation of a transcription factor. Nat Neurosci 2:339-345.
27. Riccomagno M M, Martinu L, Mulheisen M, Wu D K, Epstein D J.

2002. Specification of the mammalian cochlea is dependent on Sonic hedgehog. Genes Dev 16:2365-2378.
28. Wright T J, Hatch E P, Karabagli H, Karabagli P, Schoenwolf G C, Mansour S L. 2003. Expression of mouse fibroblast growth factor and fibroblast growth factor receptor genes during early inner ear development. Dev Dyn 228:267-272.
29. Riccomagno M M, Takada S, Epstein DJ. 2005. Wnt-dependent regulation of inner ear morphogenesis is balanced by the opposing and supporting roles of Shh. Genes Dev 19:1612-1623.
30. Ohyama T, Mohamed O A, Taketo M M, Dufort D, Groves A K. 2006. Wnt signals mediate a fate decision between otic placode and epidermis. Development 133:865-875.
31. Morsli H, Choo D, Ryan A, Johnson R, Wu D K. 1998. Development of the mouse inner ear and origin of its sensory organs. J Neurosci 18:3327-3335.
32. Ma Q, Chen Z, del Barco Barrantes I, de la Pompa J L, Anderson D J. 1998. neurogenin1 is essential for the determination of neuronal precursors for proximal cranial sensory ganglia. Neuron 20:469-482.
33. Fritzsch B, Beisel K W, Bermingham N A. 2000. Developmental evolutionary biology of the vertebrate ear: conserving mechanoelectric transduction and developmental pathways in diverging morphologies. Neuroreport 11:R35-44.
34. Fritzsch B, Beisel K W. 2001. Evolution and development of the vertebrate ear. Brain Res Bull 55:711-721.
35. Caldwell J C, Eberl D F. 2002. Towards a molecular understanding of Drosophila hearing. J Neurobiol 53:172-189.
36. Ghysen A, Dambly-Chaudiere C. 2000. A genetic programme for neuronal connectivity. Trends Genet 16:221-226.
37. Pauley S, Matei V, Beisel K W, Fritzsch B. Wiring the ear to the brain: the molecular basis of neurosensory development, differentiation, and survival. In: Kelley M K, Wu D K, Popper A N, Fay R R, editors. Development of the inner ear. New York: Springer Verlag; 2005. p 85-121.
38. Ma Q, Anderson D J, Fritzsch B. 2000. Neurogenin 1 null mutant ears develop fewer, morphologically normal hair cells in smaller sensory epithelia devoid of innervation. J Assoc Res Otolaryngol 1:129-143.
39. Matei V, Pauley S, Kaing S, Rowitch D, Beisel K W et al. 2005. Smaller inner ear sensory epithelia in Neurog 1 null mice are related to earlier hair cell cycle exit. Dev Dyn 234:633-650.
40. Ma Q, Kintner C, Anderson D J. 1996. Identification of neurogenin, a vertebrate neuronal determination gene. Cell 87:43-52.
41. Karis A, Pata I, van Doorninck J H, Grosveld F, de Zeeuw C I et al. 2001. Transcription factor GATA-3 alters pathway selection of olivocochlear neurons and affects morphogenesis of the ear. J Comp Neurol of BETA2/NeuroD1 in development of the vestibular and auditory 429:615-630.
42. Torres M, Gomez-Pardo E, Gruss P. 1996. Pax2 contributes to inner ear patterning and optic nerve trajectory. Development 122:3381-3391.
43. Pfeffer P L, Gerster T, Lun K, Brand M, Busslinger M. 1998. Characterization of three novel members of the zebrafish Pax21518 family: dependency of Pax5 and Pax8 expression on the Pax2.1 (noi) function. Development 125:3063-3074.
44. Raft S, Nowotschin S, Liao J, Morrow B E. 2004. Suppression of neural and control of inner ear morphogenesis by Tbx1. Development 131:1801-1812.
45. Hatini V, Ye X, Balas G, Lai E. 1999. Dynamics of placodal lineage development revealed by targeted transgene expression. Dev Dyn 215:332-343.
46. Pauley S, Lai E, Fritzsch B. 2006. Foxg1 is required for morphogenesis and histogenesis of the mammalian inner ear. Dev Dyn 235:2470-2482.
47. Ohyama T, Groves A K. 2004. Expression of mouse Foxi class genes in early craniofacial development. Dev Dyn 231:640-646.
48. Zou D, Silvius D, Fritzsch B, Xu P X. 2004. Eya1 and Six1 are essential for early steps of sensory neurogenesis in mammalian cranial placodes. Development 131:5561-5572.
49. Reim G, Brand M. 2002. Spiel-ohne-grenzen/pou2 mediates regional competence to respond to Fgf8 during zebrafish early neural development. Development 129:917-933.
50. Massague J, Seoane J, Wotton D. 2005. Smad transcription factors. Genes Dev 19:2783-2810.
51. Blokzijl A, ten Dijke P, Ibanez C F. 2002. Physical and functional interaction between GATA-3 and Smad3 allows TGF-beta regulation of GATA target genes. Curr Biol 12:35-45. 75.
52. Ohuchi H, Yasue A, Ono K, Sasaoka S, Tomonari S et al. 2005. Identification of cis-element regulating expression of the mouse Fgf10 gene during inner ear development. Dev Dyn 233:177-187.
53. Nicolussi A, D'Inzeo S, Santulli M, Colletta G, Coppa A. 2003. TGF-beta control of rat thyroid follicular cells differentiation. Mol Cell Endocrinol 207:1-11.
54. Seoane J, Le H V, Shen L, Anderson S A, Massague J. 2004. Integration of Smad and forkhead pathways in the control of neuroepithelial and glioblastoma cell proliferation. Cell 117:211-223.
55. Boyer L A, Lee T I, Cole M F, Johnstone S E, Levine S S et al. 2005. Core transcriptional regulatory circuitry in human embryonic stem cells. Cell 122:947-956.
56. Gowan K, Helms A W, Hunsaker T L, Collisson T, Ebert P J et al. 2001. Crossinhibitory activities of Ngn1 and Math1 allow specification of distinct dorsal interneurons. Neuron 31:219-232.
57. Zhao Y, Wang Y, Wang Z, Liu H, Shen Y et al. 2006. Sonic hedgehog promotes mouse inner ear progenitor cell proliferation and hair cell generation in vitro. Neuroreport 17:121-124.
58. Mowbray C, Hammerschmidt M, Whitfield T T. 2001. Expression of BMP signalling pathway members in the developing zebrafish inner ear and lateral line. Mech Dev 108:179-184.
59. Farinas I, Jones K R, Tessarollo L, Vigers A J, Huang E et al. 2001. Spatial shaping of cochlear innervation by temporally regulated neurotrophin expression. J Neurosci 21:6170-6180.
60. Fritzsch B, Beisel K W, Jones K, Farinas I, Maklad A et al. 2002. Development and evolution of inner ear sensory epithelia and their innervation. J Neurobiol 53:143-156.
61. Kiernan A E, Pelling A L, Leung K K, Tang A S, Bell D M et al. 2005. Sox2 is required for sensory organ development in the mammalian inner ear. Nature 434:1031-1035.
62. Radde-Gallwitz K, Pan L, Gan L, Lin X, Segil N, Chen P. 2004. Expression of Islet1 marks the sensory and neuronal lineages in the mammalian inner ear. J Comp Neurol 477:412-421.
63. Pauley S, Wright T J, Pirvola U, Ornitz D, Beisel K, Fritzsch B. 2003. Expression and function of FGF10 in mammalian inner ear development. Dev Dyn 227:203-215.
64. Kim W Y, Fritzsch B, Serls A, Bakel L A, Huang E J et al. 2001. NeuroDnull mice are deaf due to a severe loss of the inner ear sensory neurons during development. Development 128:417-426.
65. Liu M, Pereira F A, Price S D, Chu M J, Shope C et al. 2000. Essential role of BETA2/NeuroD1 in development of the vestibular and auditory systems. Genes Dev 14:2839-2854.
66. Fritzsch B, Beisel K W. 2003. Molecular conservation and novelties in vertebrate ear development. Curr Top Dev Biol 57:1-44.
67. Bermingham N A, Hassan B A, Price S D, Vollrath M A, Ben-Arie N et al. 1999. Math1: an essential gene for the generation of inner ear hair cells. Science 284:1837-1841.
68. Fritzsch B, Matei V A, Nichols D H, Bermingham N, Jones K et al. 2005. Atoh1 null mice show directed afferent fiber growth to undifferentiated ear sensory epithelia followed by incomplete fiber retention. Dev Dyn 233:570-583
69. Satoh T, Fekete D M. 2005. Clonal analysis of the relationships between mechanosensory cells and the neurons that innervate them in the chicken ear. Development 132:1687-1697.
70. Bertrand N, Castro D S, Guillemot F. 2002. Proneural genes and the specification of neural cell types. Nat Rev Neurosci 3:517-530.
71. Calof A L, Bonnin A, Crocker C, Kawauchi S, Murray R C et al. 2002. Progenitor cells of the olfactory receptor neuron lineage. Microsc Res Tech 58:176-188.
72. Beites C L, Kawauchi S, Crocker C E, Calof A L. 2005. Identification molecular regulation of neural stem cells in the olfactory epithelium. Exp Cell Res 306:309-316.
73. Kawauchi S, Beites C L, Crocker C E, Wu H H, Bonnin A et al. 2004. Molecular signals regulating proliferation of stem and progenitor cells in mouse olfactory epithelium. Dev Neurosci 26:166-180.
74. Kawauchi S, Shou J, Santos R, Hebert J M, McConnell S K et al. 2005. Fgf8 expression defines a morphogenetic center required for olfactory neurogenesis and nasal cavity development in the mouse. Development 132:5211-5223.
75. Wu H H, Ivkovic S, Murray R C, Jaramillo S, Lyons K M et al. 2003. Autoregulation of Neurogenesis by GDF11. Neuron 37:197-207.
76. Lee Y S, Liu F, Segil N. 2006. A morphogenetic wave of p27Kip1 transcription directs cell cycle exit during organ of Corti development. Development 133:2817-2826.
77. Jones J M, Montcouquiol M, Dabdoub A, Woods C, Kelley M W.
2006. Inhibitors of differentiation and DNA binding (Ids) regulate Math1 and hair cell formation during the development of the organ of Corti. J Neurosci 26:550-558.
78. Kageyama R, Ohtsuka T, Hatakeyama J, Ohsawa R. 2005. Roles of bHLH genes in neural stem cell differentiation. Exp Cell Res 306:343-348.
79. Pozniak C D, Pleasure S J. 2006. A tale of two signals: Wnt and Hedgehog in dentate neurogenesis. Sci STKE 2006:pe5.
80. Pirvola U, Spencer-Dene B, Xing-Qun L, Kettunen P, Thesleff I et al. 2000. FGF/FGFR-2(IIIb) signaling is essential for inner ear morphogenesis. J Neurosci 20:6125-6134.
81. Wright T J, Mansour S L. 2003. Fgf3 and Fgf10 are required for mouse otic placode induction. Development 130:3379-3390.
82. Ladher R K, Wright T J, Moon A M, Mansour S L, Schoenwolf G C. FGF8 initiates inner ear induction in chick and mouse. Genes Dev 19:603-613.
83. Martynoga B, Morrison H, Price D J, Mason J O. 2005. Foxg1 is required for specification of ventral telencephalon and region-specific regulation of dorsal telencephalic precursor proliferation and apoptosis. Dev Biol 283:113-127.
84. Yao J, Liu Y, Lo R, Tretjakoff I, Peterson A, Stifani S. 2000. Disrupted development of the cerebral hemispheres in transgenic mice expressing the mammalian Groucho homologue transducin-like-enhancer of split 1 in postmitotic neurons. Mech Dev 93:105-115.
85. Lanford P J, Kelley M W. Notch signaling and cell fate determination in the vertebrate inner ear. In: Kelley M W, K. W D, Popper A N, Fay R R editors. Development of the Inner Ear. Volume SHAR 26, Springer Handbook of Auditory Research. New York, N.Y.: Springer; 2005. p 122-157.
86. Lanford P J, Lan Y, Jiang R, Lindsell C, Weinmaster G et al. 1999. Notch signalling pathway mediates hair cell development in mammalian cochlea. Nat Genet 21:289-292.
87. Kiernan A E, Xu J, Gridley T. 2006. The Notch Ligand JAG1 is Required for Sensory Progenitor Development in the Mammalian Inner Ear. PLOS Genet 2:e4.
88. Brooker R, Hozumi K, Lewis J. 2006. Notch ligands with contrasting functions: Jagged1 and Delta1 in the mouse inner ear. Development 133:1277-1286.
89. Daudet N, Lewis J. 2005. Two contrasting roles for Notch activity in chick inner ear development: specification of prosensory patches and lateral inhibition of hair-cell differentiation. Development 132:541-551.
90. Ohsuka T, Imayoshi I, Shimojo H, Nishi E, Kageyama R, McConnell S K. 2006. Visualization of embryonic neural stem cells using Hes promoters in transgenic mice. Mol Cell Neurosci 31:109-122.
91. Zine A, Aubert A, Qiu J, Therianos S, Guillemot F et al. 2001. Hes1 and Hes5 activities are required for the normal development of the hair cells in the mammalian inner ear. J Neurosci 21:4712-4720.
92. Lanford P J, Shailam R, Norton C R, Gridley T, Kelley M W. 2000. Expression of Math1 and HES5 in the cochleae of wildtype and Jag2 mutant mice. J Assoc Res Otolaryngol 1:161-171.
93. Ledent V, Vervoort M. 2001. The basic helix-loop-helix protein family: comparative genomics and phylogenetic analysis. Genome Res cells. 11:754-770.
94. Westendorf J J. 2006. Transcriptional co-repressors of Runx2. J Cell Biochem 98:54-64.
95. Aronson B D, Fisher A L, Blechman K, Caudy M, Gergen J P. 1997. Groucho-dependent and -independent repression activities of Runt domain proteins. Mol Cell Biol 17:5581-5587.
96. Marcal N, Patel H, Dong Z, Belanger-Jasmin S, Hoffman B et al. 2005. Antagonistic effects of Grg6 and Groucho/TLE on the transcription repression activity of brain factor 1/FoxG1 and cortical neuron differentiation. Mol Cell Biol 25:10916-10929.
97. Mitsui K, Shirakata M, Paterson B M. 1993. Phosphorylation inhibits DNA-binding activity of MyoD homodimers but not MyoD-E12 hetero-dimers. J Biol Chem 268:24415-24420.
98. Ninkovic J, Tallafuss A, Leucht C, Topczewski J, Tannhauser B et al. 2005. Inhibition of neurogenesis at the zebrafish midbrain-hindbrain boundary by the combined and dose-dependent activity of a new hairy/E(spl) gene pair. Development 132:75-88.
99. Amoutzias G D, Weiner J, Bornberg-Bauer E. 2005. Phylogenetic profiling of protein interaction networks in eukaryotic transcription factors reveals focal proteins being ancestral to hubs. Gene 347:247-253.
100. Amoutzias G D, Robertson D L, Oliver S G, Bornberg-Bauer E. 2004. Convergent evolution of gene networks by single-gene duplications in higher eukaryotes. EMBO Rep 5:274-279.
101. Qian D, Radde-Gallwitz K, Kelly M, Tyrberg B, Kim J et al. 2006. Basic helix-loop-helix gene Hes6 delineates the sensory hair cell lineage in the inner ear. Dev Dyn 235:1689-1700.
102. Koyano-Nakagawa N, Kim J, Anderson D, Kintner C. 2000. Hes6 acts in a positive feedback loop with the neurogenins to promote neuronal differentiation. Development 127:4203-4216.
103. Kang S A, Seol J H, Kim J.?? 2005. The conserved WRPW motif of Hes6 mediates proteasomal degradation. Biochem Biophys Res Commun 332: -36.
104. Louvi A, Artavanis-Tsakonas S. 2006. Notch signalling in vertebrate neural development. Nat Rev Neurosci 7:93-102.
105. Moloney D J, Panin V M, Johnston S H, Chen J, Shao L et al. 2000. Fringe is a glycosyltransferase that modifies Notch. Nature 406:369-375.
106. Chen P, Segil N. 1999. p27(Kip1) links cell proliferation to morphogenesis in the developing organ of Corti. Development 126:1581-1590.
107. Zhang N, Martin G V, Kelley M W, Gridley T. 2000. A mutation in the Lunatic fringe gene suppresses the effects of a Jagged2 mutation on inner hair cell development in the cochlea. Curr Biol 10:659-662.
108. Yagi M, Kanzaki S, Kawamoto K, Shin B, Shah P P et al. 2000. Spiral ganglion neurons are protected from degeneration by GDNF gene therapy. J Assoc Res Otolaryngol 1:315-325.
109. Matter-Sadzinski L, Puzianowska-Kuznicka M, Hernandez J, Ballivet M, Matter J M. 2005. A bHLH transcriptional network regulating the specification of retinal ganglion cells. Development 132:3907-3921.
110. Szeder V, Grim M, Halata Z, Sieber-Blum M. 2003. Neural crest origin of mammalian Merkel cells. Dev Biol 253:258-263.
111. Hertzano R, Montcouquiol M, Rashi-Elkeles S, Elkon R, Yucel R et al. 2004. Transcription profiling of inner ears from Pou4f3(ddl/ddl) identifies Gfi1 as a target of the Pou4f3 deafness gene. Hum Mol Genet 13:2143-2153.
112. Xiang M, Maklad A, Pirvola U, Fritzsch B. 2003. Brn3c null mutant mice show long-term, incomplete retention of some afferent inner ear innervation. BMC Neurosci 4:2.
113. Haeberle H, Fujiwara M, Chuang J, Medina M M, Panditrao M V et al. 2004. Molecular profiling reveals synaptic release machinery in Merkel cells. Proc Natl Acad Sci USA 101:14503-14508.
114. Leonard J H, Cook A L, Van Gele M, Boyle G M, Inglis K J et al. 2002. Proneural and proneuroendocrine transcription factor expression in cutaneous mechanoreceptor (Merkel) cells and Merkel cell carcinoma. Int J Cancer 101:103-110.
115. Ben-Arie N, Hassan B A, Bermingham N A, Malicki D M, Armstrong D et al. 2000. Functional conservation of atonal and Math1 in the CNS and PNS. Development 127:1039-1048.
116. Cau E, Casarosa S, Guillemot F. 2002. Mash1 and Ngn1 control distinct steps of determination and differentiation in the olfactory sensory differentiation. Development 129:1871-1880.
117. Conrad R, Barrier M, Ford L P. 2006. Role of miRNA and miRNA processing factors in development and disease. Birth Defects Res C Embryo Today 78:107-117.
118. Weston M D, Pierce M L, Rocha-Sanchez S, Beisel K W, Soukup G A. 2006. MicroRNA gene expression in the mouse inner ear. Brain Res 1111:95-104.
119. Andl T, Murchison E P, Liu F, Zhang Y, Yunta-Gonzalez M et al. 2006. The miRNA-processing enzyme dicer is essential for the morphogenesis and maintenance of hair follicles. Curr Biol 16:1041-1049.
120. Fekete D M, Wu D K. 2002. Revisiting cell fate specification in the inner ear. Curr Opin Neurobiol 12:35-42.
121. Fritzsch B, Beisel K W, Hansen L A. 2006. The molecular basis of neurosensory cell formation in ear development: a blueprint for hair cell and sensory neuron regeneration? Bioessays 28:1181-1193.

What is claimed is:

1. A skin cell that comprises at least one chromosomally integrated stem cell specific gene operably linked to a promoter, wherein the promoter's activity is sensitive to the concentration of an activator.

2. The skin cell of claim 1, wherein the promoter is inactive in the absence of the activator.

3. The skin cell of claim 1, wherein the promoter's activity increases with increasing concentration of the activator.

4. The skin cell of claim 1, wherein the activator is an antibiotic.

5. The skin cell of claims 1, wherein the stem cell specific gene is selected from the group of genes comprising Oct 4(Pou5f1), Nanog, Sox2, GATA3, Neurog 1, KLF4, c-MYC, and LIN28.

6. The skin cell of claim 1, wherein the stem cell specific gene is integrated by a recombinase.

7. The skin cell of claim 6, wherein the recombinase is a phage integrase.

8. The skin cell of claim 1, wherein the cell was contacted with a composition comprising at least one first vector that comprises at least one stem cell specific gene operably linked to a promoter, wherein the promoter's activity is sensitive to the concentration of an activator and at least one second vector that comprises a recombinase.

9. A method for generating a skin cell derived stem cell, the method comprising:
a. integrating at least one stem cell specific gene into the chromosomal DNA of a skin cell, wherein the stem cell specific gene is operably linked to a promoter, wherein the promoter's activity is sensitive to the concentration of an activator, and
b. expressing the stem cell specific gene to generate a skin cell derived stem cell.

10. The method of claim 9, wherein the promoter is inactive in the absence of the activator.

11. The method of claim 9, wherein the promoter's activity increases with increasing concentrations of the activator.

12. The method of claim 9, wherein the activator is an antibiotic.

13. The method of claim 9, wherein the stem cell specific gene is selected from the group of genes consisting of Oct 4(Pou5f1), Nanog, Sox2, GATA3, Neurog 1, KLF4, c-MYC, and LIN28.

14. The method of claim 9, wherein the stem cell specific gene is integrated by a recombinase.

15. The method of claim 14, wherein the recombinase is a phage integrase.

16. The method of claim 9, wherein the stem cell is a pluripotent stem cell.

17. The method of claim 9, further comprising inducing the skin cell derived stem cell to differentiate into an ear cell by increasing the activity of Atoh1 in the skin cell derived stem cell.

18. The method of claim 17, wherein the ear cell is selected from the group of ear cells consisting of sensory neurons, hair cells, supporting cells, and non-sensory epithelial cells.

19. A method for generating an ear cell from a skin cell, the method comprising:
a. integrating at least one stem cell specific gene into the chromosomal DNA of a skin cell, wherein the stem cell specific gene is operably linked to a promoter, wherein the promoter's activity is sensitive to the concentration of an activator;
b. administering an activator to induce expression of the stem cell specific gene thereby generating a skin cell derived stem cell;
c. stopping the administration of the activator; and
d. inducing the skin cell derived stem cell to differentiate into an ear cell.

20. The method of claim 19, the method further comprising administering the ear cell generated from a skin cell derived stem cell to a subject to decrease hearing loss.
